(19) United States
(12) Patent Application Publication
(10) Pub. No.: US 2022/0365095 A1
(43) Pub. Date: Nov. 17, 2022

(54) MASS SPECTROMETRY METHOD, MASS SPECTROMETER, AND PROGRAM

(71) Applicants: SHIMADZU CORPORATION, Kyoto-shi, Kyoto (JP); TOKYO METROPOLITAN GERIATERIC HOSPITAL AND INSTITUTE OF GERONTOLOGY, Tokyo (JP)

(72) Inventors: Takashi NISHIKAZE, Kyoto-shi, Kyoto (JP); Hiroki TSUMOTO, Tokyo (JP)

(73) Assignees: SHIMADZU CORPORATION, Kyoto-shi, Kyoto (JP); TOKYO METROPOLITAN GERIATERIC HOSPITAL AND INSTITUTE OF GERONTOLOGY, Tokyo (JP)

(21) Appl. No.: 17/625,534

(22) PCT Filed: Jul. 6, 2020
(86) PCT No.: PCT/JP2020/026486

§ 371 Date: Jul. 14, 2022

(30) Foreign Application Priority Data
Jul. 23, 2019 (JP) 2019-135344

(51) Int. Cl. G01N 33/68; G01N 30/72; G01N 30/86

(52) U.S. Cl. CPC G01N 33/6848; G01N 30/7233; G01N 30/8624; G01N 2030/027

(57) ABSTRACT

A mass spectrometry method includes detecting, in a first mass spectrometry of a sample containing a glycan having a plurality of sialic acids each modified differently, a plurality of oxonium ions derived from each of the plurality of sialic acids, and calculating relative values of intensities of the plurality of oxonium ions based on data obtained by the detection.

### TECHNICAL FIELD

The present invention relates to a mass spectrometry method, a mass spectrometry apparatus, and program.

### BACKGROUND

A glycan, a glycopeptide or the like is analyzed by mass spectrometry. A sample containing a glycan often contains various molecules, and thus can be separated in multiple stages by liquid chromatography/mass spectrometry (LC/MS) or the like. Data analysis in such mass spectrometry becomes complicated. In particular, when the sample contains a glycoprotein or a glycopeptide, the sample obtained by digestion with an enzyme or the like contains a mixture of the peptide and the glycopeptide, and data analysis is more difficult. At this time, fragment ions specific to glycans are detected from mass spectra obtained by tandem mass spectrometry, and peaks corresponding to glycans are identified based on the detection.

In dissociation or the like of a glycan or a glycopeptide, an oxonium ion containing a monosaccharide, a disaccharide, a trisaccharide or the like contained in the glycan can be generated. In Patent Literature 1, oxonium ions are measured by changing an energy on collision-induced dissociation (CID), and a glycan structure is analyzed.

A glycan may contain sialic acid which is a monosaccharide much present in a living body. Sialic acid is also contained in a glycan bound to a protein in vivo, and is often present at the non-reducing end of the glycan. Therefore, sialic acid plays an important role because sialic acid is disposed outside a molecule in such a glycoprotein molecule and is directly recognized by other molecules.

Sialic acid may have different linkage types with adjacent glycans. For example, in human N-linked glycans (N-glycans), linkage types of mainly α2,3- and α2,6-, and in O-linked glycans (O-glycans) and glycosphingolipids, in addition to these, linkage types of α2,8- and α2,9-are known. Due to such different linkage types, sialic acid can be recognized from different molecules and have different roles.

In mass spectrometry or the like for glycans containing sialic acid, sialic acid is modified as pretreatment. This eliminates disadvantages such as suppression of ionization and detachment of sialic acid, by neutralizing a carboxy group of sialic acid having a negative charge by esterification, amidation, or the like. Sialic acid is easily lactonized in a glycan molecule, but stability of lactone produced varies depending on the linkage type, and therefore sialic acid can be modified and analyzed in a linkage type-specific manner using this difference in stability. The lactone is very unstable and is easily hydrolyzed even in water and is more rapidly hydrolyzed under acidic or basic conditions. Therefore, it has been reported that a lactone produced by modification in pretreatment is stabilized by amidation (see Patent Literature 2, Non Patent Literature 1, and Non Patent Literature 2).

Non Patent Literature 3 reports that a modification in which α2,3-sialic acid is amidated with ethylenediamine and α2,6-sialic acid is ethyl-esterified is performed, and oxonium ions are detected by two-stage tandem mass spectrometry (MS/MS).

### CITATION LIST

### Patent Literature

Patent Literature 1: JP 2014-66704 A
Patent Literature 2: JP 6135710 B

### Non Patent Literature

Non Patent Literature 1: Nishikaze T, Tsumoto H, Sekiya S, Iwamoto S, Miura Y, Tanaka K. “Differentiation of Sialyl Linkage Isomers by One-Pot Sialic Acid Derivatization for Mass Spectrometry-Based Glycan Profiling” Analytical Chemistry, (USA), ACS Publications, Feb. 21, 2017, Volume 89, Issue 4, pp. 2353-2360
Non Patent Literature 2: Hanamatsu H, Nishikaze T, Miura N, Piao J, Okada K, Sekiya S, Iwamoto S, Sakamoto N, Tanaka K, Furukawa JI. “Sialic Acid Linkage Specific Derivatization of Glycosphingolipid Glycans by Ring-Opening Aminolysis of Lactones” Analytical Chemistry, (USA), ACS Publications, Oct. 29, 2018, Volume 90, Issue 22, pp. 13193-13199
Non Patent Literature 3: Yang S, Wu WW, Shen RF, Bern M, Cipollo J. “Identification of Sialic Acid Linkages on Intact Glycopeptides via Differential Chemical Modification Using IntactGIG-HILIC” Journal of the American Society for Mass Spectrometry, (USA), Springer, Apr. 12, 2018, Volume 29, Issue 6, pp. 1273-1283.

### SUMMARY OF INVENTION

### Technical Problem

In the method of Non Patent Literature 3, although a glycopeptide containing modified sialic acid is subjected to CID, there is a problem in quantitativity, such that unmodified sialic acid is detected in its MS/MS spectrum. It is desirable to accurately analyze the composition of sialic acid contained in a glycan.

### Solution to Problem

A first aspect of the present invention relates to a mass spectrometry method including detecting, in a first mass spectrometry of a sample containing a glycan having a plurality of sialic acids each modified differently, a plurality of oxonium ions derived from each of the plurality of sialic acids, and calculating ratios of intensities of the plurality of oxonium ions based on data obtained by the detection.

A second aspect of the present invention relates to a mass spectrometry apparatus including a data acquisition portion configured to acquire data obtained by detecting, in a first mass spectrometry of a sample containing a glycan having a plurality of sialic acids each modified differently, a plurality of oxonium ions each derived from the plurality of sialic acids, and a calculation portion configured to calculate ratios of intensities of the plurality of oxonium ions based on the data.

A third aspect of the present invention relates to a program for making a processor perform a data acquisition process of acquiring data obtained by detecting, in a first mass spectrometry of a sample containing a glycan having a plurality of sialic acids each modified differently, a plurality of oxonium ions each derived from the plurality of sialic acids, and a calculation process of calculating ratios of intensities of the plurality of oxonium ions based on the data.

### Advantageous Effects of Invention

According to the present invention, it is possible to accurately analyze the composition of sialic acid contained in a glycan, including the difference in linkage type.

### BRIEF DESCRIPTION OF DRAWINGS

FIG. 1 is a flowchart showing a flow of a mass spectrometry method of an embodiment.

FIG. 2 is a conceptual diagram showing a schematic configuration of a mass spectrometry apparatus according to an embodiment.

FIG. 3 is a flowchart showing a flow of data analysis.

FIG. 4 is a conceptual diagram for describing provision of a program.

FIG. 5 is a conceptual diagram showing a structure of a glycopeptide detected in First example.

FIG. 6 shows extracted ion chromatograms obtained for glycopeptide A (upper stage), glycopeptide B (middle stage) and glycopeptide C (lower stage) in First example.

FIG. 7 shows mass spectra of retention times at which glycopeptide A (upper stage), glycopeptide B (middle stage) and glycopeptide C (lower stage) are eluted in First example.

FIG. 8 shows mass spectra (m/z 250 to 4000) of fragment ions of glycopeptide A (upper stage), glycopeptide B (middle stage) and glycopeptide C (lower stage) in First example.

FIG. 9 shows mass spectra (m/z 250 to 400) of fragment ions of glycopeptide A (upper stage), glycopeptide B (middle stage) and glycopeptide C (lower stage) in First example.

FIG. 10 is a conceptual diagram showing a structure of a glycopeptide detected in Second example.

FIG. 11 shows a base peak chromatogram (upper stage), and extracted ion chromatograms obtained for oxonium ions of modified α2,3-sialic acid (middle stage) and dehydrated oxonium ions (lower stage) in Second example.

FIG. 12 shows extracted ion chromatograms obtained for non-dehydrated oxonium ions of modified α2,6-sialic acid (upper stage) and dehydrated oxonium ions (lower stage) in Second example.

FIG. 13 shows mass spectra in retention times at which glycopeptide D (upper stage) and glycopeptide E (lower stage) are eluted in Second example.

FIG. 14 shows mass spectra (m/z 200 to 3000) of fragment ions of glycopeptide D (upper stage) and glycopeptide E (lower stage) in Second example.

FIG. 15 shows mass spectra (m/z 200 to 400) of fragment ions of glycopeptide D (upper stage) and glycopeptide E (lower stage) in Second example.

FIG. 16 is a conceptual diagram showing a structure of a glycan detected in Third example.

FIG. 17 is a base peak chromatogram in Third example.

FIG. 18 shows extracted ion chromatograms obtained for glycan A (upper stage), glycan B (middle stage) and glycan C (lower stage) in Third example.

FIG. 19 shows mass spectra in retention times at which glycan A (upper stage), glycan B (middle stage) and glycan C (lower stage) are eluted in Third example.

FIG. 20 shows mass spectra (m/z 200 to 3200) of fragment ions of glycan A (upper stage), glycan B (middle stage) and glycan C (lower stage) in Third example.

FIG. 21 shows mass spectra (m/z 280 to 340) of fragment ions of glycan A (upper stage), glycan B (middle stage) and glycan C (lower stage) in Third example.

FIG. 22 is a table showing candidates for the composition of glycans contained in a sample in Third example.

FIG. 23 is a table showing candidates for the composition of glycans contained in a sample in Third example.

FIG. 24 is a table showing candidates for the composition of glycans contained in a sample in Third example.

### DESCRIPTION OF EMBODIMENTS

Hereinafter, embodiments for carrying out the present invention will be described with reference to the drawings.

### First Embodiment

In the mass spectrometry method of the present embodiment, a sample containing modified sialic acid is subjected to mass spectrometry, and oxonium ions of sialic acid are detected. Based on data obtained by the detection of oxonium ions, composition of sugar contained in the glycan, a structure of the glycan or the like is analyzed.

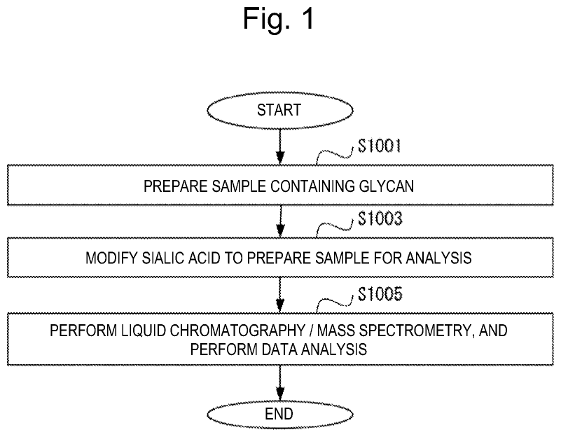

FIG. 1 is a flowchart showing a flow of a mass spectrometry method of the present embodiment. In a step S1001, a sample containing a glycan is prepared.

(Sample)

The sample containing a glycan is not particularly limited, and can contain at least one molecule selected from the group consisting of a free glycan, a glycopeptide and a glycoprotein, and a glycolipid. In particular, when the sample contains a glycopeptide or a glycoprotein, data analysis is difficult as described above, and thus it is useful to make it easier to guide the structure and the like of a glycan by the method of the present embodiment. It is preferable that the glycan in the sample contains a glycan having a possibility of having sialic acid at the terminal, such as an N-linked glycan, an O-linked glycan, or a glycolipid glycan. The glycan in the sample more preferably contains or may contain at least one of α2,3-sialic acid, α2,8-sialic acid and α2,9-sialic acid, and further preferably contains or may contain α2,6-sialic acid in addition to this.

When the sample contains a free glycan, a glycan released from a glycoprotein, a glycopeptide or a glycolipid can be used. As the releasing method, a chemical cleavage method such as enzyme treatment using N-glycosidase, O-glycosidase, endoglycoceramidase or the like, hydrazinosis or β-elimination by alkali treatment can be used. When an N-linked glycan is released from peptide chains of a glycopeptide and a glycoprotein, enzyme treatment with peptide-N-glycosidase F (PNGase F), peptide-N-glycosidase A (PNGase A), endo-β-N-acetylglucosaminidase (Endo M) or the like is suitably used. Modification such as pyridyl amination (PA) at the reducing end of the glycan can be appropriately performed. Before enzyme treatment, the peptide chain of a glycopeptide or a glycoprotein described later may be cleaved.

When the number of amino acid residues in the peptide chain of a glycopeptide or a glycoprotein is large, it is preferable to use the glycopeptide or glycoprotein after cleaving the peptide chain by enzymatic cleavage or the like. For example, in the case of preparing a sample for mass spectrometry, the number of amino acid residues of the peptide chain is preferably 30 or less, more preferably 20 or less, and further preferably 15 or less. On the other hand, when it is required to clarify the origin of the peptide to which the glycan is bonded, the number of amino acid residues of the peptide chain is preferably 2 or more, and more preferably 3 or more.

As the digestive enzyme in the case of cleaving the peptide chain of a glycopeptide or a glycoprotein, trypsin, lysyl endopeptidase, arginine endopeptidase, chymotrypsin, pepsin, thermolysin, proteinase K, pronase E or the like is used. Two or more of these digestive enzymes may be used in combination. The conditions for cleaving the peptide chain are not particularly limited, and an appropriate protocol according to the digestive enzyme to be used is adopted. Before this cleavage, denaturation treatment or alkylation treatment of the proteins and peptides in the sample may be performed. The conditions for the denaturation treatment or the alkylation treatment are not particularly limited. The peptide chain may be cleaved not by enzymatic cleavage but by chemical cleavage or the like.

After the step S1001 is completed, the process proceeds to a step S1003.

(Preparation of Sample for Analysis)

In the step S1003, sialic acid is modified to prepare a sample for analysis. The method for modifying sialic acid is not particularly limited. For example, the methods described in Patent Literature 2 and Non Patent Literature 1 described above can be used. In this method, α2,3-sialic acid, α2,8-sialic acid or α2,9-sialic acid is lactonized in a first reaction, and α2,6-sialic acid is amidated or esterified. In a second reaction, the lactonized sialic acid is modified by amidation, esterification or the like so as to form a modified product different from the modification of α2,6-sialic acid. Alternatively, the method described in Non Patent Literature 2 may be used. In this method, rapid amidation is performed by ring-opening aminolysis for directly amidating a lactone in the second reaction described above. Hereinafter, an example of performing this rapid amidation will be described. In both the first reaction and the second reaction, it is preferable that amidation is performed. This makes it possible to perform modification more stably than esterification or the like, and to analyze glycans with high accuracy.

In the step S1003, the sample prepared in the step S1001 is contacted with a reaction solution for lactonization (hereinafter, referred to as lactonization reaction solution) to perform a lactonization reaction for lactonizing at least a part of sialic acids contained in a glycan (hereinafter, when described as a lactonization reaction, it refers to the lactonization reaction in the step S1003 unless otherwise specified). In the lactonization reaction, a part of sialic acid is lactonized and the other part of sialic acid is modified differently from the lactonization in a linkage type-specific manner. In the lactonization reaction, α2,3-sialic acid, α2,8-sialic acid and α2,9-sialic acid are suitably lactonized.

The lactonization reaction solution contains a dehydration-condensation agent and a first reactant containing an alcohol, an amine or a salt thereof. The first reactant is a reactant for performing modification by esterification or amidation, by bonding at least a part of the first reactant to sialic acid. Since stability of lactone produced varies depending on the linkage type of sialic acid, the type and concentration of the dehydration-condensation agent and the first reactant are adjusted so as to selectively cause a dehydration reaction or a modification reaction by esterification or amidation based on this point. For details, refer to Patent Literature 2.

(Dehydration-Condensation Agent in Lactonization Reaction)

It is preferable that the dehydration-condensation agent contains carbodismide. This is because when carbodiimide is used, a carboxy group present at a site with high steric hindrance is less likely to be amidated than when a phosphonium-based dehydration-condensation agent (what is called BOP reagent) or an uronium-based dehydration-condensation agent is used as a dehydration-condensation agent. Examples of the carbodiimide include carbodiimides described in Patent Literature 2 described above, such as N,N′-dicyclohexylcarbodiimide (DCC), N-(3-dimethylaminopropyl)-N′-ethylcarbodiimide (EDC), or salts thereof.

(Additive in Lactonization Reaction)

In order to promote dehydration condensation by the dehydration-condensation agent and suppress a side reaction, it is preferable that a highly nucleophilic additive is used in addition to the carbodiimide. As the highly nucleophilic additive, 1-hydroxybenzotriazole (HOBt) and the like described in Patent Literature 2 described above are preferably used.

(Reactant in Lactonization Reaction (First Reactant))

It is preferable that the amine used as the first reactant contains a primary or secondary alkylamine containing two or more carbon atoms. The primary alkylamine is preferably ethylamine, propylamine, isopropylamine, butylamine, sec-butylamine, tert-butylamine, or the like. The secondary alkylamine is preferably dimethylamine, ethylmethylamine, diethylamine, propylmethylamine, isopropylmethylamine, or the like. It is preferable to use an amine having a branched alkyl group such as isopropylamine, from the viewpoint of making a carboxy group present at a site with high steric hindrance like a carboxy group of α2,3-sialic acid unlikely to be amidated. When an amine is used as the first reactant in the lactonization reaction solution, the carboxy group of a part of sialic acids such as α2,6-sialic acid is amidated based on the linkage type of the sialic acid.

The alcohol used as the first reactant is not particularly limited, and for example, methanol, ethanol or the like can be used. When an alcohol is used as the reactant of the lactonization reaction solution, the carboxy group of a part of sialic acids such as α2,6-sialic acid is esterified based on the linkage type of the sialic acid.

The first reactant may contain a salt of any of the above amines and alcohols.

(Concentration of Dehydration-Condensation Agent and Amine)

The concentration of the dehydration-condensation agent and the additive in the lactonization reaction solution can be set to, for example, 1 mM to 5 M (hereinafter, M denotes mol/L), or the like. The concentration of the amine in the lactonization reaction solution can be set to 0.01 to 20 M, or the like. The reaction temperature during the lactonization reaction can be set to about −20° C. to 100° C., or the like.

(Phase for Performing Lactonization Reaction)

The lactonization reaction can be performed in either a liquid phase or a solid phase. The state of the sample in causing the lactonization reaction is not particularly limited as long as the state can allow the sample to contact with the lactonization reaction solution.

When performing the reaction in a solid phase, the solid phase carrier can be used without particular limitation as long as the solid phase can immobilize a glycan, a glycopeptide, a glycoprotein, or the like. For example, in order to immobilize a glycopeptide or a glycoprotein, a solid phase carrier having, as a ligand, an epoxy group, a tosyl group, a carboxy group, an amino group or the like can be used. In order to capture a glycan, a solid phase carrier having, as a ligand, a hydrazide group, an aminooxy group or the like can be used. The glycan may be adsorbed on a carrier for hydrophilic interaction chromatography (HILIC).

The sample after the lactonization reaction may be subjected to treatments such as release from a solid phase carrier, purification, desalting and solubilization by a known method or the like as necessary. The same applies before and after the amidation reaction described later.

(Amidation Reaction)

As a modification reaction to be performed following the lactonization reaction, an amidation reaction is performed, in which the sample is contacted with a reaction solution (hereinafter, referred to as “amidation reaction solution”) to amidate the sialic acid lactonized in the step S1003, to acquire a sample for analysis. Conventionally, a method of ring-opening hydrolysis of a lactone followed by amidating a carboxy group with a dehydration-condensation agent has been mainly used, but a method of rapidly and directly amidating a lactone may be used. Hereinafter, ring-opening amidation of a lactone with ammonia, amine or a salt thereof is referred to as aminolysis. Since this aminolysis reaction does not substantially require a dehydration-condensation agent, it is possible to selectively amidate only lactonized sialic acid without affecting normal sialic acid not forming a lactone.

The amidation reaction solution includes a reactant (hereinafter, referred to as second reactant) containing ammonia, an amine, or a salt thereof. The second reactant is an amidation reactant for performing modification by amidation, by bonding at least a part of the second reactant to sialic acid. Preferably, the amidation reaction is performed only by contacting the sample with the amidation reaction solution, and the lactone is stabilized by a simple operation.

The amidation reaction does not require a dehydration-condensation agent, but the amidation reaction solution may contain a dehydration-condensation agent. For example, the amidation reaction solution may be prepared by adding ammonia, an amine or a salt thereof without removing the lactonization reaction solution added to the sample in the step S1003.

When the lactonization reaction solution contains the first reactant, the second reactant contained in the amidation reaction solution is different from the first reactant. The first reactant and the second reactant are selected so as to have different masses. The first reactant and the second reactant are selected according to mass resolution of mass spectrometry so that accurate mass separation is achieved for the obtained two kinds of modified products. It is preferable that the first reactant and the second reactant have different substituents for easy separation from each other through chromatography, but are not particularly limited thereto.

(Amine in Amidation Reaction)

In the following embodiments, the term “amine” includes hydrazine, hydrazine derivatives and hydroxylamine, and does not include ammonia and salts of ammonia. When an amine is used in the amidation reaction, the amine contained in the second reactant is at least one compound selected from primary amines in which one or less carbon atom is directly bonded to a carbon atom bonded to an amino group, hydrazine, hydrazine derivatives and hydroxyamines, and salts thereof. As described above, in the case of the primary amine, even if the carbon chain has a branch, if the branch is present at a position away from the amino group, a decrease in efficiency of the amidation reaction is suppressed, which is preferable.

The second reactant is more preferably a primary amine having a linear hydrocarbon group, and further preferably a primary amine having a linear alkyl group. The second reactant is, as a primary amine having a linear alkyl group, preferably a primary amine having 10 or less carbon atoms, further preferably a primary amine having 6 or less carbon atoms, that is, methylamine, ethylamine, propylamine, butylamine, pentylamine and hexylamine, and most preferably methylamine. It is preferable for the amine contained in the amidation reaction solution to have a linear structure having no branch (hereinafter, the “branch” refers to branch of a hydrocarbon chain), or have a smaller number of carbon atoms, because the lactone is more efficiently amidated. Although a polyamine such as a diamine can be used, it is preferred not to use the polyamine because the amino group remains in the modified product and oxonium ion production efficiency changes to deteriorate quantitativity.

The hydrazine derivative contained in the second reactant is not particularly limited. In the following embodiments, hydrazides such as acetohydrazide, acetic acid hydrazide, benzohydrazide and benzoic acid hydrazide are also included in the hydrazine derivative, and can be used as the second reactant. The hydrazine derivative contained in the second reactant can be at least one compound selected from the group consisting of methylhydrazine, ethylhydrazine, propylhydrazine, butylhydrazine, phenylhydrazine and benzylhydrazine, and acetohydrazide, acetic acid hydrazide, benzohydrazide and benzoic acid hydrazide. Hydrazine or a derivative thereof as the second reactant is preferably hydrazine or methylhydrazine, from the viewpoint of increasing or maintaining the efficiency of the amidation reaction.

The amine of the second reactant may contain various functional groups other than the alkyl group, such as an allyl group or a hydroxy group. When the glycan is modified so as to contain such a functional group as a result of the amidation reaction, the glycan subjected to the modification is more easily separated not only by mass spectrometry but also by chromatography or the like.

The second reactant can be ammonia and a salt of the amine described above as the second reactant.

(Concentration of Amidation Reaction Solution)

The concentration of the second reactant in the amidation reaction solution is not particularly limited, but is preferably 0.1 M or more, more preferably 0.3 M or more, further preferably 0.5 M or more, further preferably 1.0 M or more, and the most preferably 3.0 M or more. The higher the concentration of the second reactant in the amidation reaction solution, the more reliably the lactone can be amidated.

(Solvent of Amidation Reaction Solution)

The solvent of the amidation reaction solution is preferably an aqueous solvent or a mixed solvent of an aqueous solvent and an organic solvent from the viewpoint of reliably causing amidation. The solvent of the amidation reaction solution can be, for example, water, methanol, ethanol, dimethyl sulfoxide (DMSO), or an aqueous acetonitrile solution.

(pH of Amidation Reaction Solution)

The pH of the amidation reaction solution is 7.7 or more. The pH of the amidation reaction solution is preferably 8.0 or more, more preferably 8.8 or more, and further preferably 10.3 or more. When the pH of the amidation reaction solution is increased, side reactions such as hydrolysis are suppressed, and lactones are more reliably amidated using various second reactants, which is preferable.

(Time for Causing Amidation Reaction)

The amidation reaction is completed within several seconds to several minutes. Therefore, the time during which the sample is in contact with the amidation reaction solution for amidation of the lactone (hereinafter, referred to as reaction time) is preferably less than 1 hour, more preferably less than 30 minutes, further preferably less than 15 minutes, further preferably less than 5 minutes, and most preferably less than 1 minute. Preferably, it is suitable to wash the sample with the amidation reaction solution, or only to temporarily pass the amidation reaction solution through the sample held on a carrier or the like. The time during which the sample is in contact with the amidation reaction solution is not particularly limited, but can be set to appropriately 0.1 seconds or more, 1 second or more or the like, from the viewpoint of sufficiently completing the reaction, or the like. The sample may be mixed with the amidation reaction solution and directly dried and solidified without providing a reaction time. Since the amidation reaction is completed within a short time in this way, deterioration of the quantitativity due to decomposition of an unstable lactone can be prevented in analysis of glycans. By setting the reaction time of the amidation reaction to be short, the sample can be analyzed more efficiently.

(Phase for Performing Amidation Reaction)

The state of the sample in causing the amidation reaction is not limited, and may be a solid phase or a liquid phase, as long as the state can allow the sample to contact with the amidation reaction solution.

When performing the amidation reaction in a solid phase, the same solid phase carrier as that described above for the lactonization reaction can be used. For immobilization of the sample on the solid phase carrier, the conditions described above for the lactonization reaction can be used. When performing the amidation reaction in a solid phase, after the sample immobilized to the solid phase carrier is subjected to action of the amidation reaction solution for amidation, the sample is suitably released and collected from the carrier, through a chemical technique, an enzyme reaction, or the like. For example, a glycan bonded to a solid phase carrier having a hydrazide group may be liberated by a weakly acidic solution and collected. In HILIC, the amidation reaction is performed with an amidation reaction solution using acetonitrile or the like as a solvent, so that the sample can be eluted with an aqueous solution such as water.

(Suppression of Side Reaction of Glycopeptide and Glycoprotein)

When the lactonization reaction solution and the amidation reaction solution are added to a glycopeptide or a glycoprotein to modify sialic acids as described above, a side reaction may occur, such as intramolecular dehydration condensation between an amino group and a carboxy group present in the side chain of an amino acid or at a terminal of the main chain contained in the glycopeptide or glycoprotein. In this case, the side reaction of a peptide moiety in modification of sialic acids can be suppressed by preliminary blocking of amino groups by chemical modification or the like before modification of sialic acids. For details, see the following literature: Takashi Nishikaze, Sadanori Sekiya, Shinichi Iwamoto, Koichi Tanaka. “A Universal Approach to linkage-Specific Derivatization for Sialic Acids on Glycopeptides,” Journal of The American Society for Mass Spectrometry, June 2017, Volume 28, Issue 1 Supplement, Poster No. MP091. For example, a glycopeptide or a glycoprotein can be subjected to a reaction to block amino groups such as dimethylamidation and guanidinylation, followed by the lactonization reaction and the amidation reaction.

In the sample for analysis obtained by the preparation method described above, sialic acid that is of a linkage type less likely to be lactonized, such as α2,6-sialic acid, is modified with the first reactant in the lactonization reaction. Sialic acids that are of linkage types likely to be lactonized, such as α2,3-, α2,8-, and α2,9-sialic acids, are lactonized in the lactonization reaction, and modified with the second reactant in the amidation reaction.

After the step S1003 is completed, a step S1005 is started.

In the step S1005, liquid chromatography/mass spectrometry (LCMS) is performed, and the obtained data is analyzed. The sample for analysis prepared in the step S1005 is introduced into a liquid chromatograph and subjected to liquid chromatography and mass spectrometry.

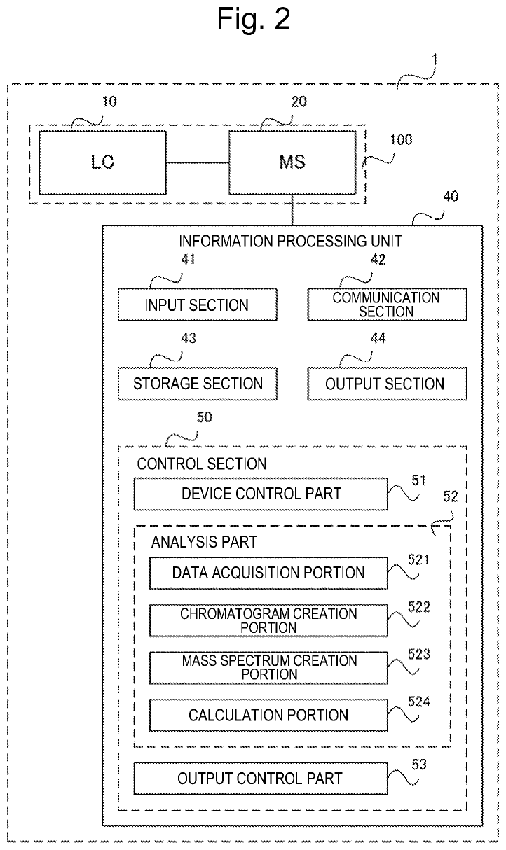

FIG. 2 is a conceptual diagram showing a configuration of a mass spectrometry apparatus according to the mass spectrometry method of the present embodiment. A mass spectrometry apparatus 1 includes a measurement unit 100 that separates and detects a sample, and an information processing unit 40. The measurement unit 100 includes a liquid chromatograph (LC) 10 and a mass spectrometer (MS) 20. The information processing unit 40 includes an input section 41, a communication section 42, a storage section 43, an output section 44, and a control section 50. The control section 50 includes a device control part 51, an analysis part 52, and an output control part 53. The analysis part 52 includes a data acquisition portion 521, a chromatogram creation portion 522, a mass spectrum creation portion 523, and a calculation portion 524.

The liquid chromatograph (LC) 10 includes an analytical column (not illustrated), and separates each component of a sample for analysis using a difference in affinity of a molecule with respect to a mobile phase and a stationary phase of the analytical column and elutes the components at different retention times. The type of the LC 10 is not particularly limited as long as each component of the sample for analysis can be separated to an extent that analysis by the analysis part 52 described later can be performed by the mass spectrometer (MS) 20. It is preferable that a molecule containing a glycan can be detected simultaneously in parallel because glycans and oxonium ions can be analyzed in association with each other. As the LC 10, nano LC, micro LC, high performance liquid chromatograph (HPLC), ultra high performance liquid chromatograph (UPLC) or the like can be used.

The type of the solution constituting the mobile phase of liquid chromatography is not particularly limited as long as each component of the sample for analysis can be separated to an extent that analysis by the analysis part 52 described later can be performed. For example, a first mobile phase contains water as a solvent, a second mobile phase contains acetonitrile as a solvent, and an additive such as formic acid may be appropriately added to these mobile phases. The first and second mobile phases are mixed based on a gradient program stored in the storage section 43 or the like and introduced into the analytical column.

The type of the analytical column of liquid chromatography is not particularly limited as long as each component of the sample for analysis can be separated to an extent that analysis by the analysis part 52 described later can be performed. The analytical column is preferably, for example, a reversed-phase column from the viewpoint of ease of handling or ease of ionization in mass spectrometry. The stationary phase of the analytical column is preferably, for example, a silane bonded with a linear hydrocarbon such as C18 supported on a carrier such as silica gel.

The sample eluted from the analytical column of the LC 10 is introduced into the mass spectrometer 20. It is preferable that the sample eluted from the LC 10 is input to the mass spectrometer 20 by online control without requiring an operation such as dispensing by a user of the mass spectrometry apparatus 1 (hereinafter, simply referred to as “user”).

(Mass Spectrometry)

The mass spectrometer 20 performs mass spectrometry on the sample introduced from the LC 10 to detect oxonium ions derived from sialic acid of a glycan contained in the sample. The method of mass spectrometry is not particularly limited as long as a plurality of oxonium ions derived from a plurality of sialic acids modified differently contained in the glycan can be detected with desired accuracy.

The mass spectrometry may be performed by tandem mass spectrometry (MSn) in which mass separation is performed in three or more stages, in addition to tandem mass spectrometry (MSIMS) in which mass separation is performed in two stages. Alternatively, single mass spectrometry with one-stage mass separation using in-source dissociation may be performed. The type of mass spectrometry is not particularly limited as long as low mass cut off does not occur in mass separation of oxonium ions. Compared with a sample such as a glycan or a glycopeptide, oxonium ions detected have a smaller mass. Therefore, it may be difficult to detect oxonium ions in some ion trap mass spectrometers in which low mass cutoff occurs. Mass spectrometry in which low mass cutoff does not occur can be performed by combining one or more arbitrary types of mass spectrometry such as quadrupole type, time-of-flight type, and ion trap type. The mass spectrometer 20 can include one or more mass spectrometers corresponding to these mass spectrometry in combination. An electric field type Fourier transform mass spectrometer called Orbitrap may be used.

When performing tandem mass spectrometry, it is preferable to detect oxonium ions by product ion scan or selected reaction monitoring (SRM). In these methods, after a molecule containing a glycan such as a glycan or a glycopeptide is ionized, a part of the generated ions is mass-separated as precursor ions. The precursor ions are subjected to dissociation to generate fragment ions (also referred to as product ions). Fragment ions derived from the glycan containing sialic acid modified in the step S1003 include oxonium ions derived from sialic acid. The generated fragment ions are subjected to mass separation and then detected by an ion detector. During mass separation of fragment ions, m/z (corresponding to mass-to-charge ratio) is scanned in product ion scan, and mass separation is performed by m/z of oxonium ions without scanning in SRM.

Data obtained by detection of ions in mass spectrometry is referred to as measurement data. In the product ion scan, in a mass spectrum of fragment ions obtained from the measurement data, peaks corresponding to a plurality of oxonium ions derived from a plurality of sialic acids modified differently are shown on the same mass spectrum, so that peaks of a plurality of oxonium ions can be displayed in an easy-to-understand manner. In SRM, it is possible to calculate intensity with high quantitativity. Other than these, any method can be performed as long as the plurality of oxonium ions can be quantitatively detected. Even in mass spectrometry other than tandem mass spectrometry, fragment ions containing oxonium ions derived from sialic acid are generated by dissociation or the like of a molecule containing a glycan, and the oxonium ions are mass-separated and detected.

The method of ionization in mass spectrometry is not particularly limited as long as the molecule containing a glycan is ionized to an extent that oxonium ions can be detected with desired accuracy. When liquid chromatography/tandem mass spectrometry (LC/MS/MS) is performed, an electrospray (ESI) method, a nano-electrospray ionization (nano-ESI) method or the like can be used. The ionization can be performed in a positive ion mode or the like.

The method of dissociation in mass spectrometry is not particularly limited as long as oxonium ions derived from sialic acid are generated, and for example, collision-induced dissociation (CID) or the like can be performed. CID can be performed by a collision cell or the like installed in the mass spectrometer 20.

The “oxonium ions derived from sialic acid” refer to oxonium ions containing at least a part of sialic acids contained in the glycan. In particular, the “oxonium ions derived from sialic acid” refer to not only oxonium ions corresponding to sialic acid as a monosaccharide but also oxonium ions containing at least one sialic acid and corresponding to a plurality of sugars bonded to each other in the glycan. The “oxonium ions derived from sialic acid” include oxonium ions corresponding to monosaccharides, disaccharides, trisaccharides, or the like. The “oxonium ions derived from sialic acid” include ions obtained by dehydration of one or more water molecules from oxonium ions corresponding to monosaccharides, disaccharides, trisaccharide, or the like (hereinafter referred to as dehydrated oxonium ions). As appropriate, oxonium ions corresponding to undehydrated monosaccharides, disaccharides, trisaccharides or the like are called non-dehydrated oxonium ions to be distinguished.

The oxonium ions detected by mass spectrometry contain at least a part of the modified product of sialic acid modification in the step S1003. This makes it possible to facilitate analysis of glycans based on the mass change assumed by modification. As a non-limiting example, when sialic acid is methylamidated, a non-dehydrated oxonium ion at m/z 305 or a dehydrated oxonium ion at m/z 287 obtained by dehydration from the non-dehydrated oxonium ion, corresponding to sialic acid, can be detected (both are omitted after decimal point of m/z, and the same applies hereinafter). When sialic acid is isopropylamidated, a non-dehydrated oxonium ion at m/z 333 or a dehydrated oxonium ion at m/z 315 obtained by dehydration from the non-dehydrated oxonium ion, corresponding to sialic acid, can be detected. In the detection of these oxonium ions, an allowable error range (tolerance) of m/z of, for example, 0.1% or less or 1% or less is set in consideration of the accuracy of mass spectrometry.

Specifically, mass spectrometry can be performed as follows. First, the sample eluted from the LC is subjected to a full scan in which the molecule containing a glycan is detected by scanning m/z and performing mass separation once. Data corresponding to the mass spectrum (hereinafter, referred to as MS1 spectrum) is created from the measurement data obtained by the full scan. Tandem mass spectrometry is performed on a peak in the MS1 spectrum. By this tandem mass spectrometry, a mass spectrum (hereinafter, referred to as MS/MS spectrum) obtained by detecting fragment ions generated by dissociation of ions corresponding to the peak is obtained. This tandem mass spectrometry corresponds to the mass spectrometry in which the detection of oxonium ions is performed. The selection of the peak in the MS1 spectrum may be performed by an analyst, or may be automatically performed by the mass spectrometry apparatus 1 as called data dependent mass spectrometry (ddMS). When information obtained in advance for glycans containing sialic acid to be analyzed is little, tandem mass spectrometry of each peak is comprehensively performed for a wide retention time and m/z range. If there is information obtained in advance, it is possible to narrow a range in which a peak to perform tandem mass spectrometry is present by using the information such as retention time or m/z.

In the data analysis in mass spectrometry, ratios of intensities of a plurality of oxonium ions corresponding to each of a plurality of sialic acids modified to form different modified products in the step S1003 are calculated based on the data obtained by mass spectrometry. The intensity of oxonium ion refers to a value indicating magnitude of a detection signal of the oxonium ion. For example, this value is calculated as an area of a peak (peak area) corresponding to an oxonium ion or a maximum intensity at the peak (peak intensity) in a mass spectrum or a chromatogram. When sialic acid is modified in a linkage type-specific manner as described above, relative values of intensities of a plurality of oxonium ions corresponding to a plurality of sialic acids modified differently depending on the linkage type of sialic acid are calculated. The “relative value” may be expressed in any form as long as the relative amounts of a plurality of sialic acids are shown. For example, the ratio may be expressed in the form of a ratio such as A:B, and a ratio of intensity of one sialic acid to intensity of the other sialic acid may be calculated.

The relative values of intensities of the plurality of oxonium ions reflect a ratio of the number of sialic acids modified differently in the molecule of the glycan. Therefore, in the data analysis in mass spectrometry, a ratio of the number of sialic acids having different linkage types contained in the glycan to be analyzed can be calculated based on the above ratio. The structure of a glycan can be estimated by a predetermined algorithm based on information of m/z of a glycan or information of mass spectra of fragment ions of detected glycans. In such an algorithm, for example, a plurality of glycan structure candidates satisfying conditions such as m/z of detected glycans are searched based on mass of each sugar that can constitute a glycan. By selecting one that satisfies the above ratio from these candidates, the composition of sugar constituting the glycan or the number of sialic acids in each linkage type contained in the glycan can be calculated.

When the data analysis described above is performed, a retention time at which a glycan containing modified sialic acid is eluted from LC 10 may be unknown, and a peak corresponding to the glycan may not be estimated. In this case, an extracted ion chromatogram (XIC) of oxonium ions derived from modified sialic acid, and information on the retention time can be acquired based on the XIC.

In creation of data corresponding to XIC, as described above, in the MS/MS spectrum corresponding to each peak in a certain range of the retention time and m/z, a peak within an error range based on the accuracy of mass spectrometry is extracted from m/z of oxonium ions, and the retention time is associated with the intensity of the extracted peak at the retention time. The retention time corresponding to the extracted peak is acquired as a retention time at which a glycan or a glycopeptide having sialic acid is eluted (hereinafter, referred to as glycan elution time), and is stored in the storage section 43 or the like.

(Operation of Information Processing Unit 40)

Data analysis in the mass spectrometry may be performed by an information processing device (not illustrated) that has acquired measurement data from the mass spectrometry apparatus 1 via communication or the like, but an example performed by the information processing unit 40 will be described below.

The information processing unit 40 includes an information processing device such as an electronic computer, and appropriately serves as an interface with a user and performs processing such as communication, storage, calculation and the like regarding various data. The information processing unit 40 controls the LC 10 and the mass spectrometer 20, and performs analysis and display processing.

The information processing unit 40 may be configured as one device integrated with the LC 10 or the mass spectrometer 20. A part of the data used in the mass spectrometry method of the present embodiment may be stored in a remote server or the like.

The input section 41 of the information processing unit 40 includes an input device such as a mouse, a keyboard, various buttons, or a touch panel. The input section 41 receives information and the like necessary for the processing performed by the control section 50 from a user. The communication section 42 of the information processing unit 40 includes a communication device capable of communicating by wireless or wired connection via a network such as the Internet. The communication section 42 receives data necessary for measurement by the measurement unit 100, transmits data processed by the control section 50, and transmits and receives necessary data as appropriate.

The storage section 43 of the information processing unit 40 includes a nonvolatile storage medium. The storage section 43 stores measurement data output from the measurement unit 100, a program for the control section 50 to execute processing, and the like. The output section 44 of the information processing unit 40 is controlled by the output control part 53 and includes a display device such as a liquid crystal monitor or a printer, and displays information regarding the measurement by the measurement unit 100, data obtained by processing of the analysis part 52 and the like on the display device or prints and outputs the data on a print medium.

The control section 50 of the information processing unit 40 includes a processor such as a CPU. The control section 50 performs various processing by executing a program stored in the storage section 43, such as control of the measurement unit 100 or analysis of measurement data output from the measurement unit 100.

The device control part 51 of the control section 50 controls measurement operations such as liquid chromatography and mass spectrometry of the measurement unit 100, based on analysis conditions or the like set according to an input or the like via the input section 41.

The analysis part 52 performs the above-described data analysis based on the measurement data.

The data acquisition portion 521 of the analysis part 52 acquires measurement data. The data acquisition portion 521 acquires measurement data output from an ion detector of the mass spectrometer 20, and stores the measurement data in a memory, the storage section 43 or the like so as to be referable from the CPU of the control section 50.

The chromatogram creation portion 522 of the analysis part 52 creates data corresponding to chromatogram (hereinafter, referred to as chromatogram data) from the measurement data. In the chromatogram data, the retention time of detected ions and the detection intensity are associated with each other. The chromatogram creation portion 522 stores the created chromatogram data in the storage section 43 or the like.

From the measurement data obtained by the full scan, the chromatogram creation portion 522 can create data corresponding to a chromatogram showing a peak corresponding to the molecule containing a glycan, which is not dissociated, such as a base peak chromatogram. Here, the base peak chromatogram is a chromatogram in which a peak having the highest peak intensity is extracted when performing the full scan for each retention time, and the peak intensity is indicated in association with the retention time.

In a case where the XIC is created, the chromatogram creation portion 522 creates data corresponding to the XIC (XIC data) from the measurement data obtained by a second mass spectrometry. The chromatogram creation portion 522 can appropriately create data corresponding to various chromatograms according to the purpose of data analysis to be performed or the like.

The mass spectrum creation portion 523 of the analysis part 52 creates data corresponding to the mass spectrum (hereinafter, referred to as mass spectrum data) from the measurement data. In the mass spectrum data, m/z of detected ions and the detection intensity are associated with each other. The mass spectrum creation portion 523 stores the created mass spectrum data in the storage section 43 or the like.

The mass spectrum creation portion 523 creates data corresponding to the mass spectrum at each elution time from the measurement data obtained by the full scan. In addition, data corresponding to a mass spectrum (MS/MS spectrum) of fragment ions is created from measurement data obtained by tandem mass spectrometry. The mass spectrum creation portion 523 can appropriately create data corresponding to various mass spectra according to the purpose of data analysis to be performed or the like.

The calculation portion 524 of the analysis part 52 calculates relative values of intensities of the plurality of oxonium ions derived from each of the plurality of sialic acids in which different modified products are formed. The calculation portion 524 refers to m/z of the plurality of oxonium ions stored in the storage section 43 or the like. The calculation portion 524 identifies each peak corresponding to the referred m/z in the MS/MS spectrum as a peak corresponding to an oxonium ion. At this time, when a glycan elution time is obtained by XIC, a peak corresponding to an oxonium ion can be identified from an MS/MS spectrum at the glycan elution time. The calculation portion 524 calculates an intensity of these oxonium ions from the peak intensity or the peak area. The calculation portion 524 calculates a relative value of the obtained intensity ratio or the like.

For example, it is assumed that α2,3-sialic acid, α2,8-sialic acid and α2,9-sialic acid are modified so as to form a first modified product, and α2,6-sialic acid is modified so as to form a second modified product different from the first modified product. In this case, the calculation portion 524 calculates a ratio between the intensity of oxonium ions based on sialic acid in which the first modified product is formed and the intensity of oxonium ions in which the second modified product is formed. Based on the calculated ratio, the ratio of the number of α2,3-sialic acid. α2,8-sialic acid and α2,9-sialic acid to the number of α2,6-sialic acid in the composition of glycans containing sialic acid can be estimated. Sensitivity of oxonium ions to be detected may vary depending on the modification method or the like, but for example, a ratio of intensities of oxonium ions of a glycan or a glycopeptide containing α2,3-sialic acid and α2,6-sialic acid one by one can be acquired in advance, stored in the storage section 43, and corrected based on the ratio. The calculation portion 524 can estimate the number of α2,3-sialic acid, α2,8-sialic acid and α2,9-sialic acid and the number of α2,6-sialic acid in the composition of glycans containing sialic acid, further based on information on mass of glycans containing sialic acid, a general structure of a glycan, or the like. Similarly, the calculation portion 524 can estimate the composition of sugar in the glycans containing sialic acid. The calculation portion 524 stores information such as the calculated relative value in the storage section 43 or the like.

The output control part 53 creates an output image including the chromatogram or mass spectrum described above, information obtained by the processing of the calculation portion 524, or the like, and causes the output section 44 to output the output image.

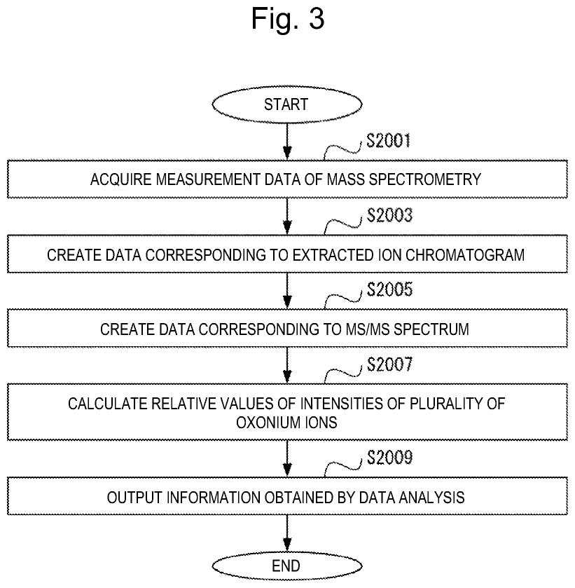

FIG. 3 is a flowchart showing a flow of data analysis of a mass spectrometry method of the present embodiment. In the flowchart of FIG. 3, an example in the case of creating XIC is shown, but the glycan elution time may be obtained by a method other than XIC. In a step S2001, the data acquisition portion 521 acquires measurement data in mass spectrometry obtained by detection of oxonium ions. After the step S2001 is completed, a step S2003 is started. In the step S2003, the chromatogram creation portion 522 creates data corresponding to the extracted ion chromatogram. The glycan elution time is acquired from the extracted ion chromatogram of oxonium ions. After the step S2003 is completed, a step S2005 is started.

In the step S2005, the mass spectrum creation portion 523 creates data corresponding to the MS/MS spectrum. From the measurement data of mass spectrometry, an MS/MS spectrum including a peak corresponding to an oxonium ion is created. After the step S2005 is completed, a step S2007 is started.

In the step S2007, the calculation portion 524 calculates relative values of intensities of the plurality of oxonium ions derived from each of the plurality of sialic acids in which different modified products are formed. After the step S2007 is completed, a step S2009 is started. In the step S2009, the output control part 53 outputs information obtained by the data analysis. After the step S2009 is completed, the processing is completed.

The following modifications are also within the scope of the present invention and can be combined with the above-described embodiment. In the following modifications, parts and the like indicating the same structures and functions as those in the above-described embodiment will be referred to by the same reference numerals, and their description will be omitted as appropriate.

(First Modification)

In the above-described embodiment, at least one piece of information including a glycan elution time, a value of mass of a glycan or a glycopeptide as a precursor and the like may be obtained by performing precursor ion scan on the sample eluted from the LC 10. In this case, the full scan may not be performed. The precursor ion scan is preferably performed by a triple quadrupole mass spectrometer.

In the precursor ion scan, m/z of precursor ions for mass separation is scanned in mass separation of a first stage. The mass-separated precursor ions are subjected to dissociation by CID or the like to generate fragment ions. From the generated fragment ions, oxonium ions derived from the modified sialic acid are mass-separated and detected based on the m/z set, in mass separation of a second stage. For example, in the second stage, non-dehydrated oxonium ions or dehydrated oxonium ions listed in the non-limiting example in the mass spectrometry described above can be mass-separated and detected.

In the data analysis in the precursor ion scan, a chromatogram in which the retention time and the detection intensity of the detected ions are made to correspond to each other is created for m/z of each detected oxonium ion. The retention time corresponding to the peak of the chromatogram is the glycan elution time. The value of m/z extracted in the first step when oxonium ions are detected in the second step is m/z of a glycan, a glycopeptide or the like containing the oxonium ions. The obtained information such as the glycan elution time and the m/z is appropriately stored in the storage section 43 or the like.

(Second Modification)

In the above embodiment, the sample has been analyzed by LC/MS, but liquid chromatography may not be performed. For example, the sample for analysis prepared in the step S1003 may be ionized by matrix-assisted laser desorption/ionization (MALDI) or the like, and oxonium ions may be generated and detected by dissociation. In particular, when information about the structure of a glycan contained in the sample is obtained, when the number of types of molecules contained in the sample is small, or the like, the structure of a glycan can be analyzed even without performing liquid chromatography.

(Third Modification)

A program for realizing information processing function of the mass spectrometry apparatus 1 may be recorded in a computer-readable recording medium, and the program regarding the control of the processing of the analysis part 52 described above and processing related to the above processing recorded in the recording medium may be read and caused to execute by a computer system. The “computer system” herein includes an operating system (OS) and hardware of peripheral devices. The “computer-readable recording medium” refers to a portable recording medium such as a flexible disk, a magneto-optical disk, an optical disk or a memory card, and a storage device such as a hard disk built in a computer system. The “computer-readable recording medium” may include a medium that dynamically holds a program for a short time, such as a communication line in a case where the program is transmitted via a network such as the Internet or a communication line such as a telephone line, and a medium that holds a program for a certain period of time, such as a volatile memory inside a computer system serving as a server or a client in that case. The program described above may be for realizing a part of the functions described above, and the functions described above may be realized by a combination with a program already recorded in the computer system.

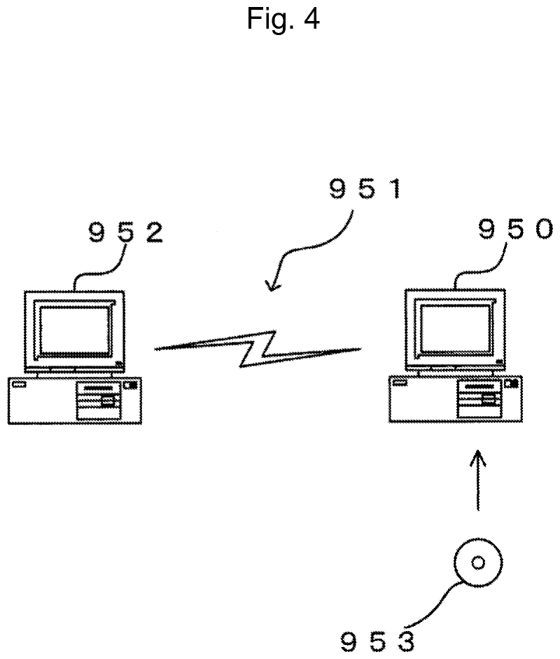

In the case of being applied to a personal computer (hereinafter, referred to as PC) or the like, the program related to the control described above can be provided through a recording medium such as a CD-ROM or a DVD-ROM or a data signal such as the Internet. FIG. 4 is a diagram showing this state. A PC 950 is provided with a program via a CD-ROM 953. The PC 950 has a connection function with a communication line 951. A computer 952 is a server computer that provides the program, and stores the program in a recording medium such as a hard disk. The communication line 951 is a communication line such as the Internet or personal computer communication, a dedicated communication line, or the like. The computer 952 reads the program using a hard disk, and transmits the program to the PC 950 via the communication line 951. That is, the program is carried as a data signal by a carrier wave and transmitted via the communication line 951. As described above, the program can be supplied as various forms of computer readable computer program products such as a recording medium and a carrier wave.

(Modes)

It is understood by those skilled in the art that the plurality of exemplary embodiments or their modifications described above are specific examples of the following modes.

(Clause 1) A mass spectrometry method according to an aspect includes detecting, in a first mass spectrometry of a sample containing a glycan having a plurality of sialic acids each modified differently, a plurality of oxonium ions derived from each of the plurality of sialic acids, and calculating relative values of intensities of the plurality of oxonium ions based on data obtained by the detection. This makes it possible to accurately analyze the composition of sialic acid contained in the glycan.

(Clause 2) In a mass spectrometry method according to another mode, in the mass spectrometry method according to Clause 1, the plurality of sialic acids are amide-modified. This makes it possible to more accurately analyze the composition of sialic acid contained in the glycan.

(Clause 3) In a mass spectrometry method according to another mode, in the mass spectrometry method according to Clause 1 or 2, the first mass spectrometry is performed by tandem mass spectrometry in two or more stages. This makes it possible to generate oxonium ions even without performing in-source dissociation.

(Clause 4) In a mass spectrometry method according to another mode, the mass spectrometry method according to any one of Clauses 1 to 3 includes preparing a sample containing a glycan having sialic acid, and modifying a plurality of sialic acids with each different linkage type contained in the glycan in a linkage type-specific manner, in which the first mass spectrometry of the sample containing the glycan having the plurality of modified sialic acids is performed. This makes it possible to accurately analyze the linkage type of sialic acid contained in the glycan.

(Clause 5) In a mass spectrometry method according to another mode, in the mass spectrometry method according to Clause 4, α2,3-sialic acid, α2,8-sialic acid or α2,9-sialic acid, and α2,6-sialic acid are each modified differently. This makes it possible to accurately analyze sialic acids in these linkage types contained in the glycan.

(Clause 6) In a mass spectrometry method according to another mode, the mass spectrometry method according to Clause 4 or 5 includes calculating a ratio of a number of a plurality of sialic acids having different linkage types in the glycan contained in the sample based on the relative values. This makes it possible to accurately obtain the ratio of the number of sialic acids in each linkage type contained in the glycan.

(Clause 7) In a mass spectrometry method according to another mode, the mass spectrometry method according to any one of Clauses 1 to 6 includes performing chromatography of the sample before the first mass spectrometry. This makes it possible to more accurately analyze the composition of sialic acid contained in the glycan by separation by chromatography.

(Clause 8) In a mass spectrometry method according to another mode, the mass spectrometry method according to Clause 7 includes outputting an extracted ion chromatogram including a peak corresponding to at least one of the plurality of oxonium ions. This makes it possible to clearly show the time during which the glycan containing sialic acid is eluted.

(Clause 9) In a mass spectrometry method according to another mode, the mass spectrometry method according to Clause 7 or 8 includes performing mass separation of ions generated by ionization of the sample based on scanned m/z, performing dissociation of the mass-separated ions, and performing second mass spectrometry for detecting oxonium ions from the ions generated by the dissociation, and obtaining at least one of a time during which a molecule containing a glycan from which the detected oxonium ion is derived is eluted in the chromatography and a mass of the molecule, based on a result of the second mass spectrometry. This makes it possible to more easily identify a peak corresponding to the glycan containing sialic acid.

(Clause 10) A mass spectrometry apparatus according to one mode includes a data acquisition portion configured to acquire data obtained by detecting, in a first mass spectrometry of a sample containing a glycan having a plurality of sialic acids each modified differently, a plurality of oxonium ions each derived from the plurality of sialic acids, and a calculation portion configured to calculate relative values of intensities of the plurality of oxonium ions based on the data. This makes it possible to accurately analyze the composition of sialic acid contained in the glycan.

(Clause 11) A program according to one mode is for making a processor perform a data acquisition process (corresponding to step S2005 in the flowchart of FIG. 3) of acquiring data obtained by detecting, in a first mass spectrometry of a sample containing a glycan having a plurality of sialic acids each modified differently, a plurality of oxonium ions each derived from the plurality of sialic acids, and a calculation process (corresponding to step S2009) of calculating relative values of intensities of the plurality of oxonium ions based on the data. This makes it possible to accurately analyze the composition of sialic acid contained in the glycan.

The present invention is not limited to the contents of the above embodiments. Other aspects conceivable within the scope of the technical idea of the present invention are also included in the scope of the present invention.

### Example

In the following examples, an example in which oxonium ions are detected by mass spectrometry after the linkage type-specific modification of sialic acid contained in a glycopeptide or a glycan is performed will be described. In the following linkage type-specific modification, α2,3-sialic acid was lactonized and then methylamidated, and α2,6-sialic acid was isopropylamidated. In the following Examples, the horizontal axis of chromatogram represents retention time, and the vertical axis represents a relative amount in which intensity of detected ion is represented by relative value. The horizontal axis of mass spectrum represents m/z, and the vertical axis represents the relative amount.

The present invention is not limited to amide modification aspects, numerical values, conditions or the like shown in the following Examples.

In First example and Second example, a glycoprotein was digested to obtain a glycopeptide, and then a sample obtained by purifying the glycopeptide was subjected to LC/MS. Pretreatment and LC/MS conditions are as follows.

<Digestion of Glycoprotein>

The purchased commercially available glycoprotein was reacted in the presence of 6 M (M denotes mol/L) urea, 50 mM ammonium bicarbonate, and 5 mM tris(2-carboxyethyl)phosphine hydrochloride (TCEP) at room temperature for 45 minutes to perform denaturation and reduction. Subsequently, the reacted glycoprotein was reacted in the presence of 10 mM iodoacetamide (IAA) at room temperature under light-shielding conditions for 45 minutes to perform alkylation, and then reacted in the presence of 10 mM dithiothreitol (DTT) at room temperature under light-shielding conditions for 45 minutes to deactivate excess IAA. Thereafter, trypsin was added to the glycoprotein after the reaction, and the mixture was reacted overnight at 37° C. to perform protease digestion. After protease digestion, a sample containing the obtained digest was desalted using a desalting carrier, and dried and solidified with SpeedVac (Thermo Fisher Scientific).

<Dimethylation of Amino Group>

In order to suppress the side reaction described above, the amino group of the glycopeptide or glycoprotein was previously blocked by chemical modification. To the dried and solidified glycoprotein digest was added 20 μL of a 100 mM triethylammonium bicarbonate (TEAB) buffer solution (pH 8.5). The digest was dissolved using a vortex mixer, 1.6 μL of a 2% aqueous formaldehyde solution was then added to the solution, and the mixture was gently mixed using a vortex mixer and spun down. To the solution after spinning down was added 1.6 μL of a 300 mM aqueous sodium cyanoborohydride solution, and the mixture was reacted at room temperature for 1 hour while being gently mixed using a vortex mixer. To the reacted solution was added 3.2 μL of 1% aqueous ammonia to quench the mixture, and the mixture was gently mixed using a vortex mixer and spun down. To the solution after spinning down was added 1.6 μL of formic acid, and the mixture was gently mixed and spun down using a vortex mixer. Then, 72 μL of water was added to the mixture, and desalting purification and removal of excess reagent were performed using a desalting carrier.

<Linkage Type-Specific Modification of Sialic Acid>

To the dimethylated glycoprotein digest was added 20 μL of a sialic acid linkage type-specific amidation reaction solution (2 M isopropylamine hydrochloride (iPA-HCl), 500 mM N-(3-dimethylaminopropyl)-N′-ethylcarbodiimide hydrochloride (EDC-HCl), 500 mM 1-hydroxybenzotriazole (HOBt), solvent:dimethyl sulfoxide (DMSO)), and the mixture was reacted at normal temperature for 1 hour while being stirred at 2000 rpm. To the reacted solution was added 20 μL of a 10% aqueous methylamine solution as an amidation reaction solution, and the mixture was stirred with a vortex mixer. To the reacted solution was added 160 μL of an acetonitrile (ACN) solution containing 2.5% trifluoroacetic acid (TFA) to make a total of 200 μL, then the mixture was subjected to amide purification.

<Amide Purification>

100 μL of H₂O was added to an amide chip (GL Sciences Inc.), and then discharged by centrifugation. Then, the same operation was sequentially performed using 90% ACN. Thereafter, the reaction solution diluted with ACN was added to the amide chip, and the solution was discharged by centrifugation. Further, after adding 150 μL of 90% ACN to the amide chip, discharge by centrifugation was repeated twice, and washing was performed. Finally, 20 μL of H₂O was added to the amide chip, and discharge by centrifugation was repeated twice to elute the glycopeptide. The two eluates were combined, and the solvent was removed by SpeedVac and dried and solidified. Glycopeptide concentration was performed with removal of excess reagent.

<LC/MS>

The sample obtained by amide purification was dissolved in a liquid and subjected to LC/MS. LC/MS conditions were as follows.

Liquid Chromatography Conditions

The sample for analysis was separated by liquid chromatography under the following conditions.

System: Ultimate 3000 RSLCnano (Thermo Fisher Scientific)

Trap column: C18 PepMap 100 (inner diameter 0.3 mm, length 5 mm, particle size 5 μm, Thermo Fisher Scientific)

Analytical column: NTCC-360/75-3-125 (Nikkyo Technos)

Column temperature: 35.0° C.

Mobile phase:
(A) 0.1% formic acid (dissolved in water)
(B) 0.1% formic acid (dissolved in acetonitrile)

Flow rate: 300 nL/min

Gradient program:

| | Concentration of |
|---|---|
| Time (min) | mobile phase B (%) |
| 0 | 2.0 |
| 3.0 | 2.0 |
| 18.0 | 40.0 |
| 20.0 | 95.0 |
| 30.0 | 95.0 |
| 32.0 | 2.0 |
| 45.0 | 2.0 |

Mass Spectrometry Conditions

The elution sample eluted in the liquid chromatography was detected by a quadrupole-electric field type Fourier transform mass spectrometer.

### System: Q Exactive (Thermo Fisher Scientific)

Ionization method: Nanoelectrospray method, positive ion mode

Mass spectrometry was performed by data-dependent MS (dd MS). In dd MS, a mass spectrum (MS1 spectrum) of an elution sample ionized by full scan was obtained. Thereafter, precursor ions were selected using m/z corresponding to a peak having high intensity in the MS1 spectrum, and product ion scan was performed to obtain a mass spectrum of fragment ions (hereinafter, referred to as MS2 spectrum). In First example, an extracted ion chromatogram was created from measurement data obtained by the full scan, for m/z of the assumed glycopeptide. In Second example, a base peak chromatogram was created from the data obtained by the full scan. In Second example, an extracted ion chromatogram was created based on the m/z of oxonium ions derived from modified sialic acid appearing in the MS2 spectrum automatically acquired depending on data.

### First Example

In First example, α1-acid glycoprotein (AGP) as a glycoprotein was used as a sample, and pretreatment such as digestion of the glycoprotein was performed as described above, and the following glycopeptides A, B and C were detected from the obtained glycopeptides by LC/MS.

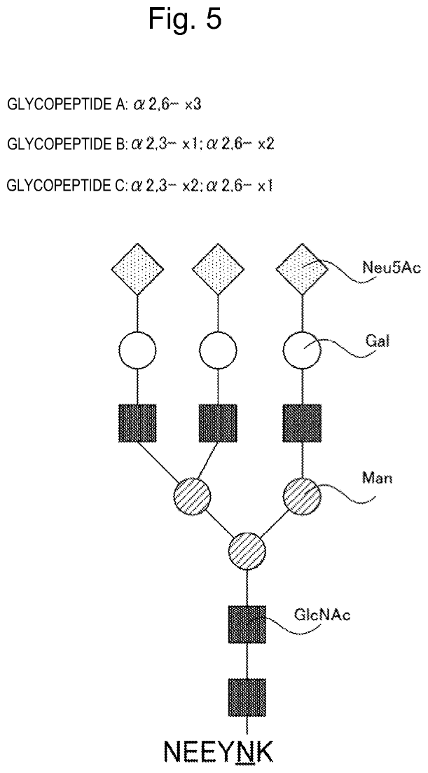

FIG. 5 is a conceptual diagram showing a structure common to glycopeptides A, B and C analyzed in this example. In glycopeptides A, B and C, the sequence of the peptide moiety is NEEYNK (SEQ ID NO: 1) in a single character code, and a three-chain trisialyl glycan is bonded to asparagine. This glycan has a basic structure composed of N-acetyl-D-glucosamine (GlcNAc) and mannose (Man), and three side chains. GlcNAc, galactose (Gal) and sialic acid (Neu5Ac) are bonded to the three side chains, respectively. Glycopeptide A contains three α2,6-sialic acids. Glycopeptide B contains one α2,3-sialic acid and two α2,6-sialic acids. Glycopeptide C contains two α2,3-sialic acids and two α2,6-sialic acids.

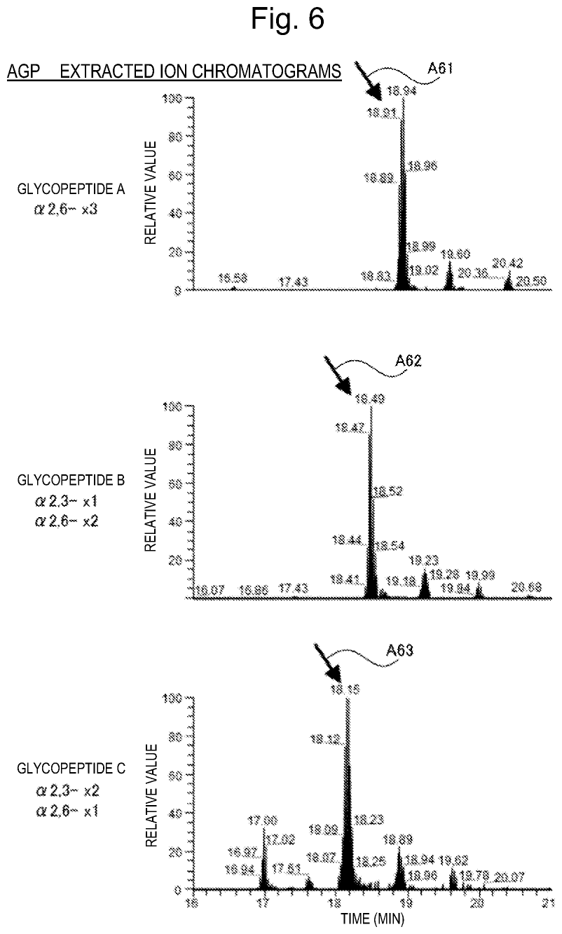

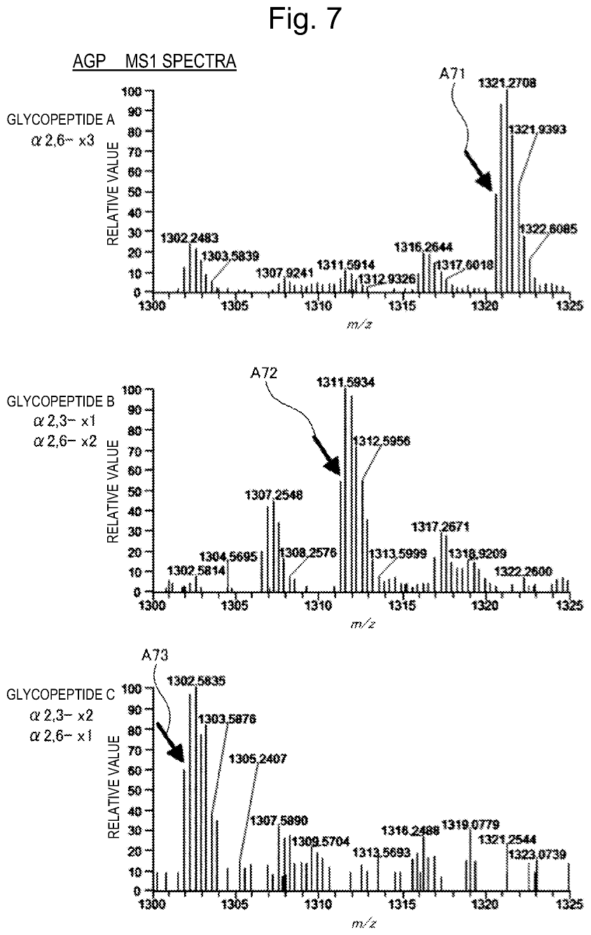

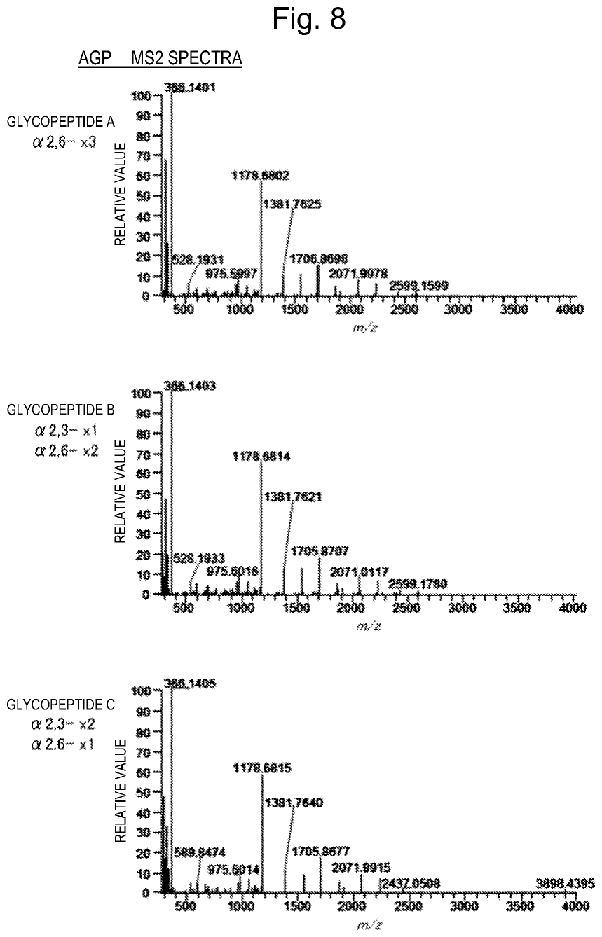

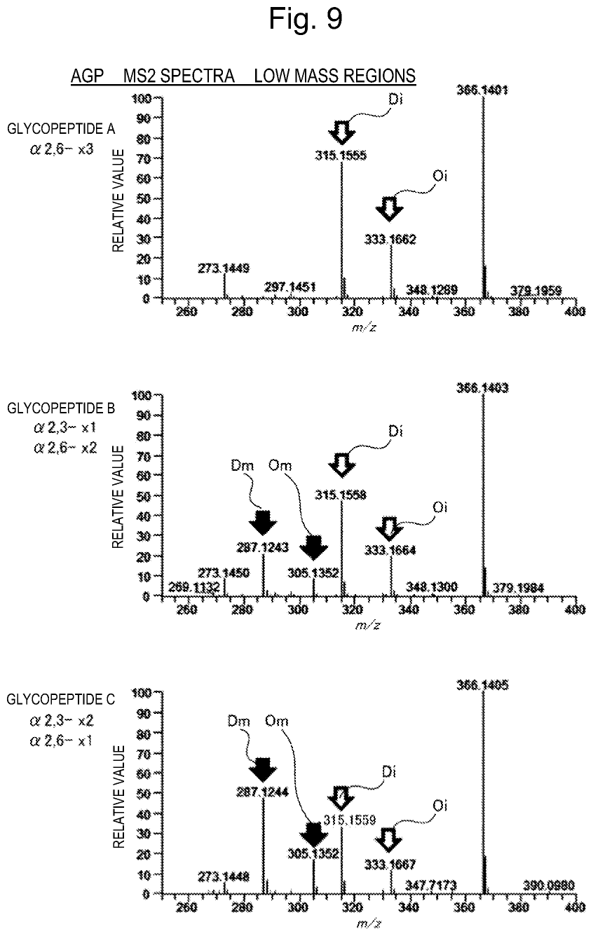

FIG. 6 is a diagram showing extracted ion chromatograms of glycopeptide A (upper stage), glycopeptide B (middle stage) and glycopeptide C (lower stage) obtained in this example. FIG. 7 shows MS1 spectra of glycopeptide A (upper stage), glycopeptide B (middle stage) and glycopeptide C (lower stage) in retention times indicated by arrows A61, A62 and A63 in FIG. 6, respectively. FIG. 8 shows MS2 spectra of glycopeptide A (upper stage), glycopeptide B (middle stage) and glycopeptide C (lower stage), in which ions corresponding to the peaks indicated by arrows A71, A72 and A73 in FIG. 7, respectively, are precursor ions. FIG. 9 shows MS2 spectra in which low mass regions (m/z 250 to 400) of the MS2 spectra of FIG. 8 are enlarged, for glycopeptide A (upper stage), glycopeptide B (middle stage) and glycopeptide C (lower stage). In FIG. 9, Oi represents a peak corresponding to non-dehydrated oxonium ions derived from isopropylamidated sialic acid, Di represents a peak corresponding to dehydrated oxonium ions derived from isopropylamidated sialic acid, Om represents a peak corresponding to non-dehydrated oxonium ions derived from methylamidated sialic acid, and Dm represents a peak corresponding to dehydrated oxonium ions derived from methylamidated sialic acid, and the same applies to the following figures.

At different retention times, a peak corresponding to each glycopeptide with a difference of 28 Da by monovalent conversion was observed (FIG. 6). The difference of 28 Da corresponds to a difference obtained by converting the difference between α2,3 and α2,6 into a mass difference of sialic acid by linkage type-specific modification. When MS2 spectra (FIG. 8) at these peaks are compared, it is found that m/z of fragment ions not containing sialic acid and their patterns are similar, and it is found that only the linkage type of sialic acid is different.

In the MS2 spectra (FIG. 9) showing the enlarged low m/z regions, non-dehydrated oxonium ions (m/z 305) and dehydrated oxonium ions (m/z 287) derived from methylamidated α2,3-sialic acid by linkage type-specific modification, and non-dehydrated oxonium ions (m/z 333) and dehydrated oxonium ions (m/z 315) derived from isopropylamidated α2,6-sialic acid were observed. Ions corresponding to the peaks Oi and Di at m/z 333 and m/z 315 were observed from glycopeptide A containing only α2,6-sialic acid, and ions corresponding to the peaks Om and Dm at m/z 305 and m/z 287 were also observed from glycopeptides B and C containing both α2,3-sialic acid and α2,6-sialic acid. This shows that different oxonium ions are generated depending on the linkage type of sialic acid. The ratio of oxonium ions derived from α2,3-sialic acid and oxonium ions derived from α2,6-sialic acid roughly reflects the α2,3-/α2,6-ratio contained in the precursor ions.

Identification of glycopeptides becomes more reliable by using structural information of a glycan obtained in this example. So far, it has not been clear what kind of oxonium ions are generated from amidated sialic acid. However, by using the information obtained by the detection of oxonium ions of the amidated sialic acid as structural information, it is possible to exclude a molecule not containing sialic acid as a candidate for a glycopeptide or a molecule not containing sialic acid that is of a linkage type suggested from the mass of oxonium ion. Furthermore, even when both α2,3-sialic acid and α2,6-sialic acid are contained in the glycan, it is possible to exclude those in which the content ratio of α2,3-sialic acid and α2,6-sialic acid is far from the intensity ratio of oxonium ions. As a result, it is possible to more easily analyze the structure of glycopeptide than before.

### Second Example

In Second example, haptoglobin (HPT) as a glycoprotein was used as a sample, and pretreatment such as digestion of the glycoprotein was performed as described above, and the following glycopeptides D and E were detected from the obtained glycopeptides by LC/MS.

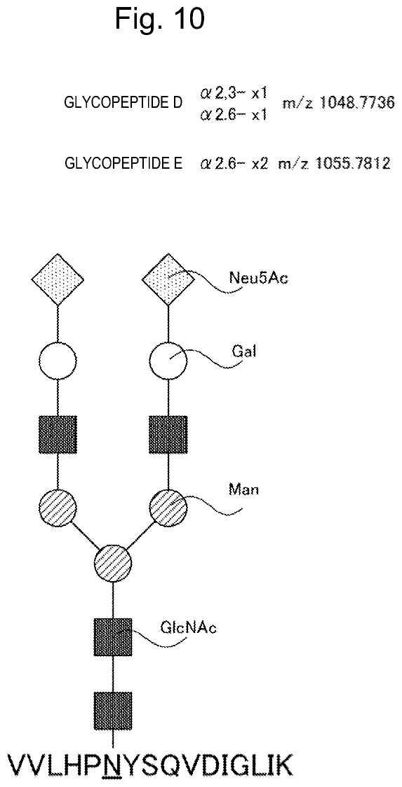

FIG. 10 is a conceptual diagram showing a structure common to glycopeptides D and E analyzed in this example. In glycopeptides D and E, the sequence of the peptide moiety is VVLHPNYSQVDIGLIK (SEQ ID NO: 2) in a single character code, and a glycan is bonded to asparagine. This glycan has a basic structure composed of GlcNAc and Man and two side chains. GlcNAc, Gal and sialic acid (Neu5Ac) are bonded to the two side chains, respectively. Glycopeptide D contains one α2,3-sialic acid and one α2,6-sialic acid. Glycopeptide E contains two α2,6-sialic acids.

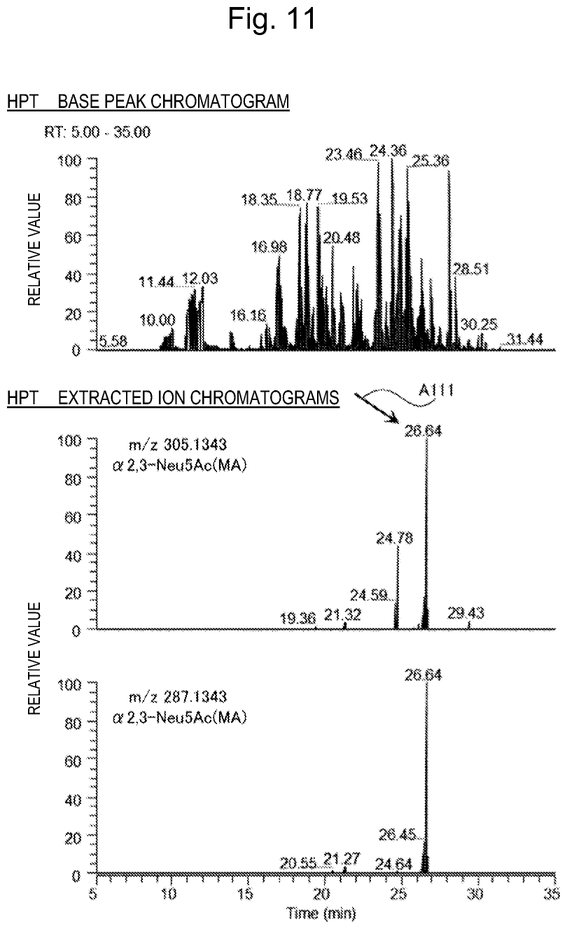

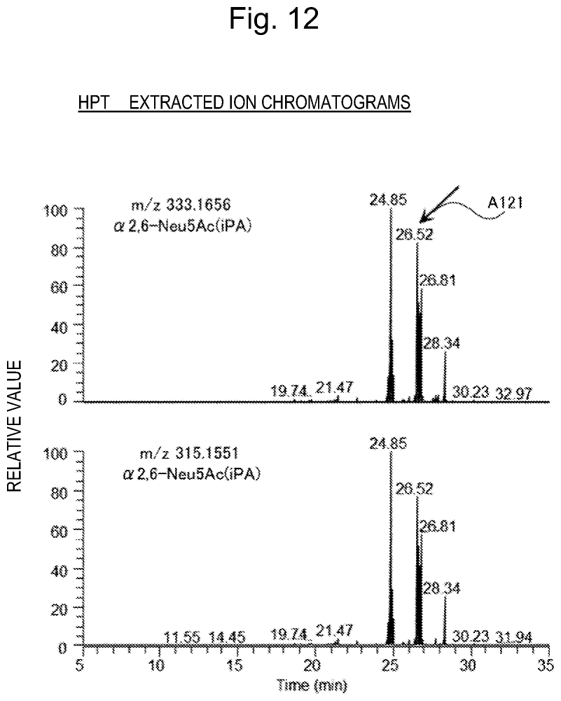

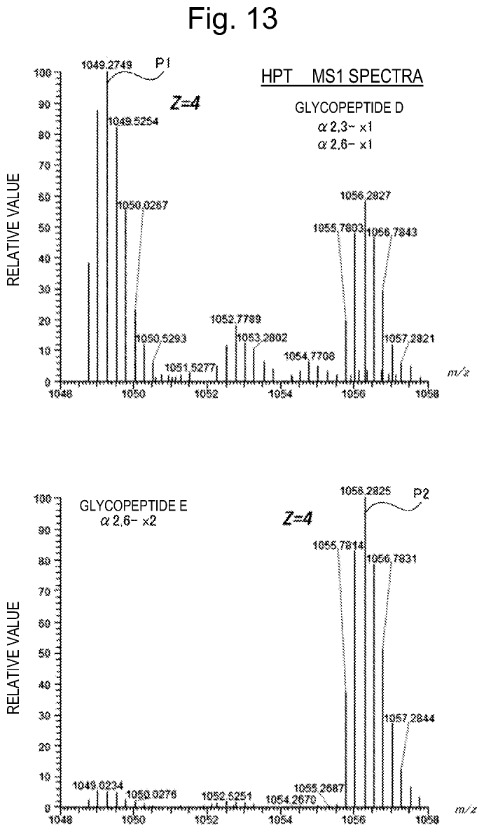

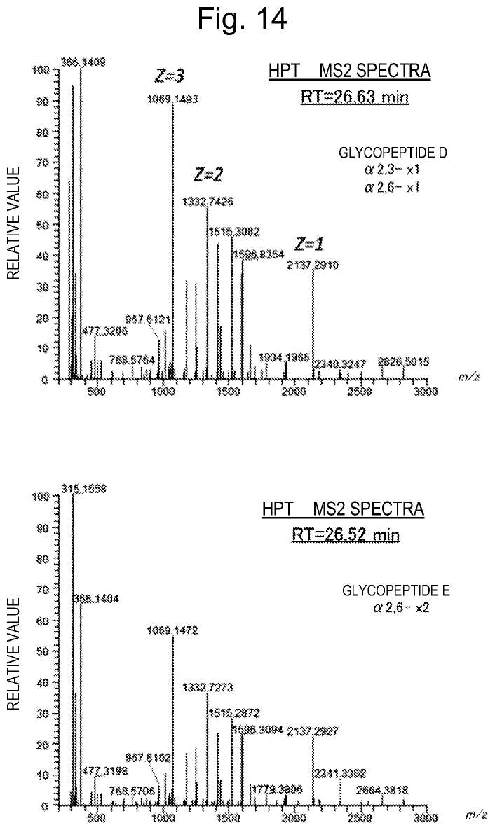

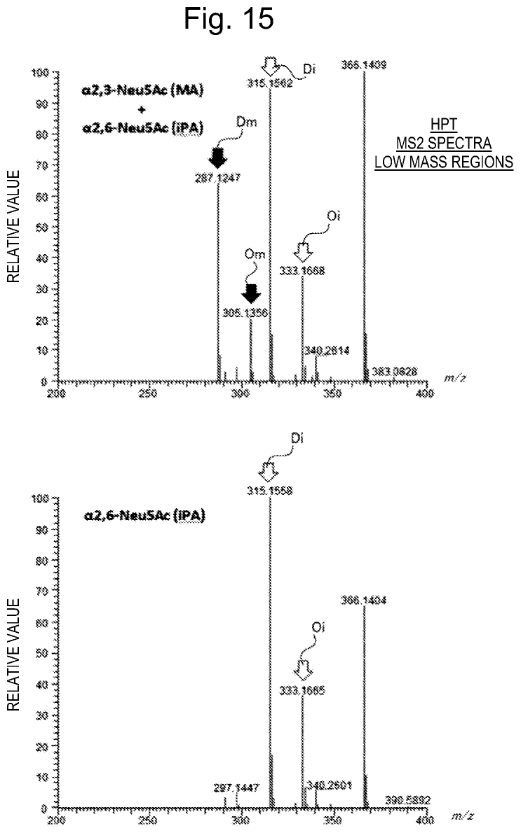

FIG. 11 is a diagram showing a base peak chromatogram (upper stage), and extracted ion chromatograms of non-dehydrated oxonium ions derived from methylamidated sialic acid (middle stage) and dehydrated oxonium ions derived from methylamidated sialic acid (lower stage) obtained in this example. FIG. 12 is a diagram showing extracted ion chromatograms of non-dehydrated oxonium ions derived from isopropylamidated sialic acid (upper stage) and dehydrated oxonium ions derived from isopropylamidated sialic acid (lower stage) obtained in this example. FIG. 13 shows MS1 spectra of glycopeptide D (upper stage) and glycopeptide E (lower stage) in retention times indicated by arrow A111 in FIG. 11 and arrow A121 in FIG. 12, respectively. FIG. 14 shows MS2 spectra of glycopeptide D (upper stage) and glycopeptide E (lower stage), in which ions corresponding to the peaks P1 and P2 in FIG. 13, respectively, are precursor ions. FIG. 15 shows MS2 spectra in which low mass regions (m/z 200 to 400) of the MS2 spectra of FIG. 14 are enlarged, for glycopeptide D (upper stage) and glycopeptide E (lower stage).

As shown in FIG. 11 (middle and lower stages) and FIG. 12, by drawing an extracted ion chromatogram (XIC) with specific fragment ions, it is possible to extract precursor ions having specific fragments from a huge number of precursor ions as displayed in the base peak chromatogram (upper stage of FIG. 11) and visually display them. When XIC was drawn at m/z 305 and 287 respectively corresponding to the non-dehydrated oxonium ions and the dehydrated oxonium ions derived from methylamidated sialic acid, elution position of the glycopeptide with α2,3-sialic acid was shown. When XIC was drawn at m/z 333 and 315 respectively corresponding to the non-dehydrated oxonium ions and the dehydrated oxonium ions derived from isopropylamidated sialic acid, elution position of the glycopeptide with α2,6-sialic acid was shown. Even if XIC of oxonium ions derived from sialic acid is displayed in a state in which sialic acid modification is not performed or in a state in which linkage type non-specific modification is performed on sialic acid, it is merely to display elution position of the glycopeptide or glycan having sialic acid. However, by drawing XIC for the mass of oxonium ions based on the modification after performing linkage type-specific modification on sialic acid as described above, it is possible to distinguish precursor ions having sialic acid having a specific linkage type and visually display them.

Detected monoisotopic masses (Mm) of glycopeptide D and glycopeptide E shown in FIG. 10 are Mm 4191.0641 (glycopeptide D) and Mm 4219.0965 (glycopeptide E) by monovalent conversion (FIG. 13: “Z=4” in FIG. 13 represents a valence of the ion), and a mass difference of 28 Da corresponds to a mass difference between isopropylamidated sialic acid and methylamidated sialic acid. In the MS2 spectra of glycopeptide D and glycopeptide E (FIG. 14), the masses and patterns of fragment ions not containing sialic acid were almost the same, and it could be confirmed that the glycopeptides were different only in the sialic acid linkage type. The glycopeptides contain two sialic acids, but oxonium ions of modified sialic acid are observed in the low m/z regions of the MS2 spectra of glycopeptide D and glycopeptide E, and it is found that glycopeptide D contains both α2,3-sialic acid and α2,6-sialic acid one by one, and glycopeptide E contains only α2,6-sialic acid. As can be seen from FIGS. 9 and 15, it was observed that the oxonium ion of α2,6-sialic acid modified with isopropylamine tended to have a stronger peak than the oxonium ion of α2,3-sialic acid modified with methylamine.

### Third Example

In Third example, the following glycans A, B and C were detected by LC/MS from a sample containing glycans released from glycoproteins, and analyzed.

<Release of N-Linked Glycan from Serum-Derived Glycoprotein>

Glycoproteins contained in 4 μL of serum were denatured and reduced in the presence of SDS and DTT, NP-40 was added, then PNGaseF was added, and the mixture was incubated overnight at 37° C. to release N-linked glycans.

<Sialic Acid Modification on Hydrazide Beads>

Samples containing N-linked glycans released from serum were bound to hydrazide beads (BlotGlyco, manufactured by Sumitomo Bakelite Co., Ltd.). The binding method followed a standard protocol of BlotGlyco. After binding of the glycans, excess hydrazide groups on the beads were capped with acetic anhydride according to the standard protocol. Thereafter, the beads were washed three times with 200 μL of DMSO, 100 μL of a sialic acid linkage type-specific amidation reaction solution (2 M iPA-HCl, 500 mM EDC-HCl, 500 mM HOBt, solvent: DMSO) was added to the washed beads, and the mixture was reacted at normal temperature for 1 hour while being lightly stirred at 800 rpm, and the reaction solution was removed by centrifugation. The beads were then washed three times with 200 μL of methanol (MeOH). After washing, 100 μL of a 10% aqueous methylamine solution was added as an amidation reaction solution, and the mixture was gently stirred and centrifuged to remove the reaction solution. After washing with 200 μL of water three times, 20 μL of water and 180 μL of 2% acetic acid/acetonitrile were added, and the mixture was heated on a heat block at 75° C. for 90 minutes to release the glycans from the beads. The released glycans were collected by washing with 50 μL of water, and those obtained by repeating collection by washing were combined as an eluate and centrifugally dried and solidified under reduced pressure.

<Labeling of Glycans with 2-Aminobenzoic Acid (2AA)>

5 μL of a 2AA-labeling reaction solution (30% acetic acid/DMSO 100 μL containing 5 mg 2AA, 6 mg sodium cyanoborohydride) was added to the dried and solidified N-linked glycans, and the glycans were well redissolved, then the solution was reacted on a heat block at 65° C. for 2 hours. After the reaction, the reaction solution was diluted with 95 μL of acetonitrile, and the excess reagent was removed using the amide chip (manufactured by GL Sciences Inc.).

<LC/MS>

The obtained sample was subjected to LC/MS. The LC/MS conditions are the same as the analysis conditions of First example and Second example. However, in this example, an extracted ion chromatogram was created based on the m/z of the assumed glycan.

<Results>

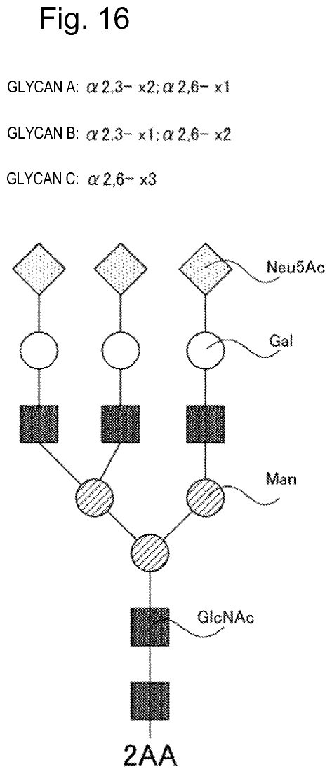

FIG. 16 is a conceptual diagram showing a structure common to glycans A, B and C analyzed in this example. Glycans A, B and C have the same structure as the glycan moiety of the glycopeptide used in First example, but are different in that the reducing end is labeled with 2AA. Glycan A contains three α2,6-sialic acids. Glycan B contains one α2,3-sialic acid and two α2,6-sialic acids. Glycan C contains two α2,3-sialic acids and two α2,6-sialic acids.

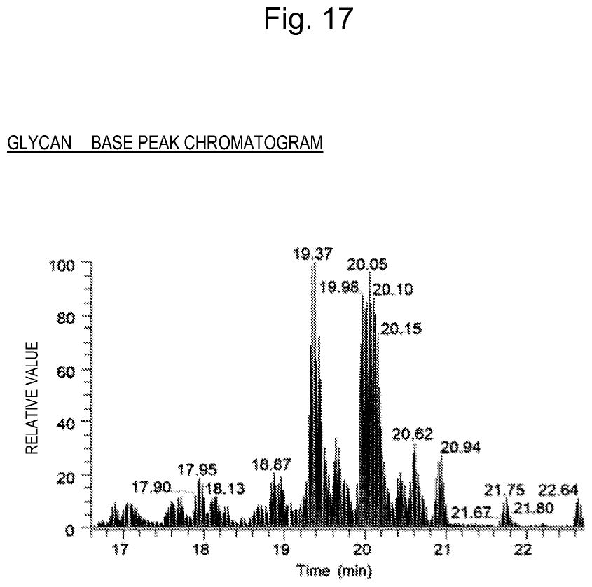

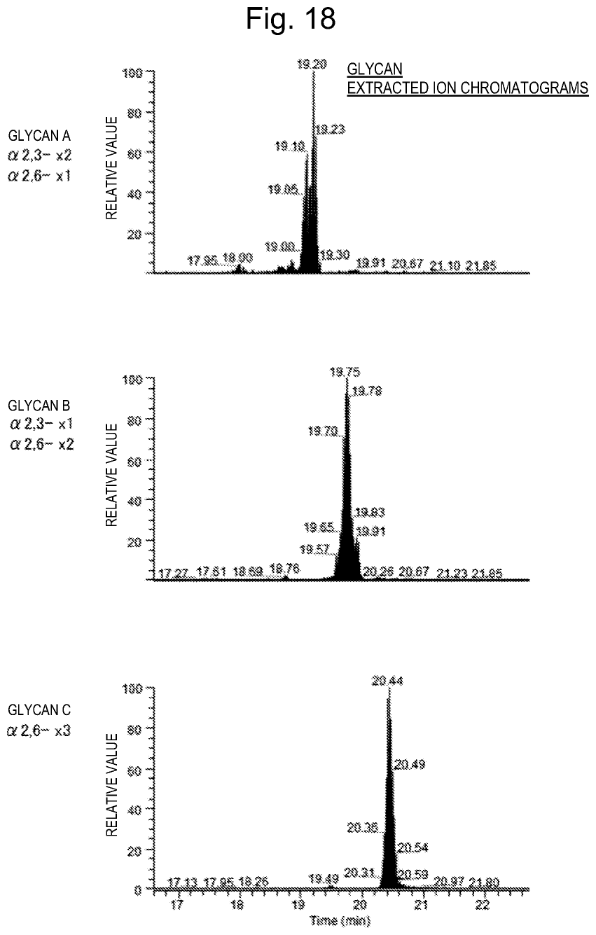

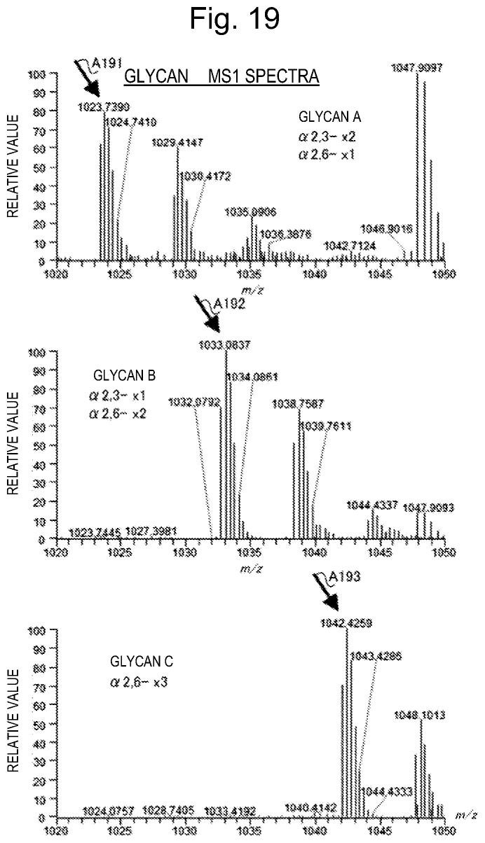

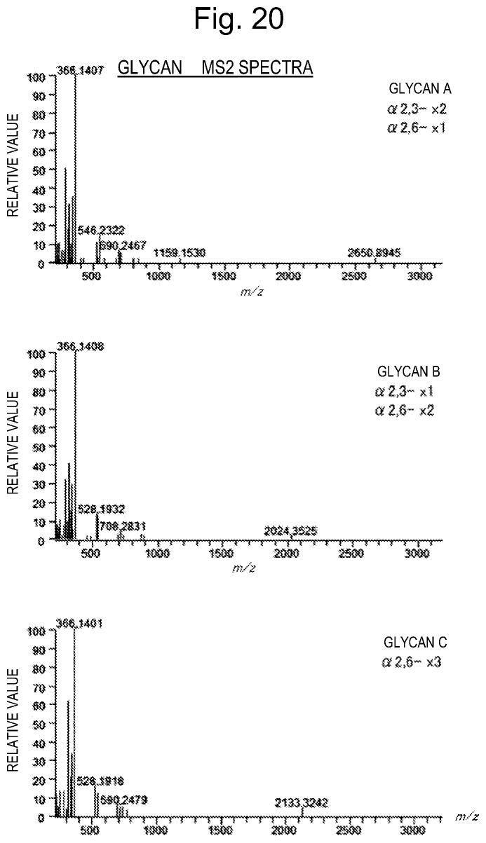

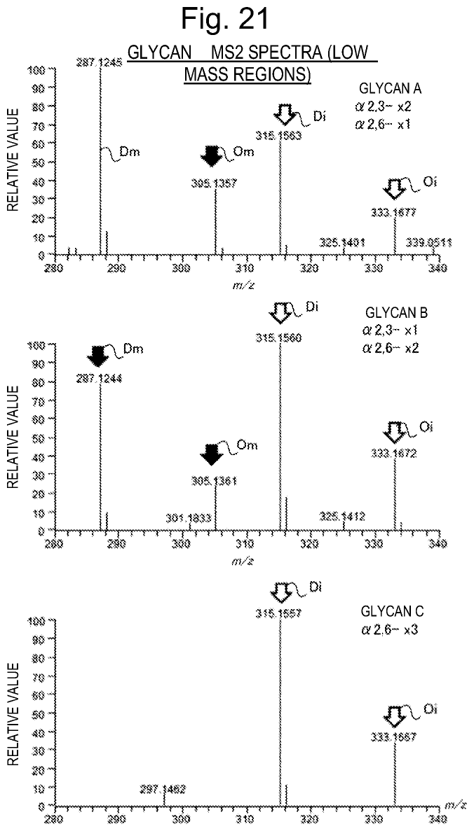

FIG. 17 is a diagram showing a base peak chromatogram obtained in this Example. FIG. 18 shows extracted ion chromatograms of glycan A (upper stage), glycan B (middle stage) and glycan C (lower stage). FIG. 19 shows MS1 spectra of glycan A (upper stage), glycan B (middle stage) and glycan C (lower stage). FIG. 20 shows MS2 spectra of glycan A (upper stage), glycan B (middle stage) and glycan C (lower stage), in which ions corresponding to the peaks indicated by arrows A191, A192 and A193 in FIG. 19, respectively, are precursor ions. FIG. 21 shows MS2 spectra in which low mass regions (m/z 280 to 340) of the MS2 spectra of FIG. 20 are enlarged, for glycan A (upper stage), glycan B (middle stage) and glycan C (lower stage).

From the base peak chromatogram shown in FIG. 17, three types, glycans A, B and C, have been detected as ions corresponding to the three-chain glycans in FIG. 16, and it is found that, referring to MS1 spectra (FIG. 19), the ions have been detected as triply-charged form of [M+3H]+. MS2 spectra (FIG. 20) of glycans A, B and C showed similar patterns. However, in the MS2 spectra in the low m/z regions (FIG. 21), oxonium ions Oi and Di of isopropylamidated sialic acid and oxonium ions Om and Dm of methylamidated sialic acid have been detected, and the linkage type of sialic acid contained in the glycan and the ratio of α2,3-sialic acid/α2,6-sialic acid can be estimated from the presence/absence and the intensity ratio of these oxonium ions.

<Analysis of Structure of Glycan>

An example of analyzing the structure of glycan A will be described below. For m/z 1023.4057 (triply-charged) of the detected glycans, possible glycan composition candidates were calculated with software created by the applicant. By setting the number and type of each monosaccharide, the software mechanically calculates a combination of these monosaccharides in a brute-force manner, and displays a combination of monosaccharides that falls within a tolerance range set by the user on the software from the detected m/z. The search conditions for the glycan composition candidates were hexose (Hex) 3 to 10, N-acetylhexosamine (HexNAc) 2 to 10, deoxyhexose (dHex) 0 to 3, N-acetylneuraminic acid (NeuAc) 0 to 4, N-glycolylneuraminic acid (NeuGc) 0 to 4, and sulfation modification (Sulfation) 0 to 1, with numbers as the numbers of monosaccharides. The tolerance for m/z of the detected glycan was 0.2 Da, and the mass change by the modification method of this example was taken into consideration as sialic acid modification.

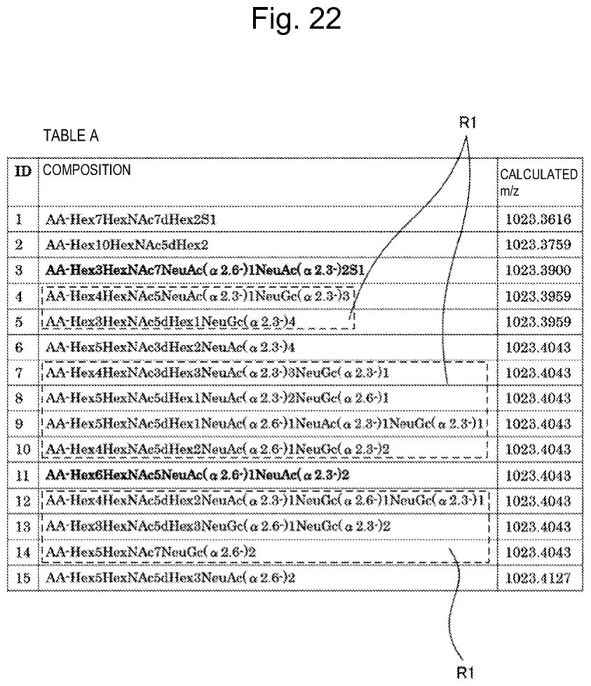

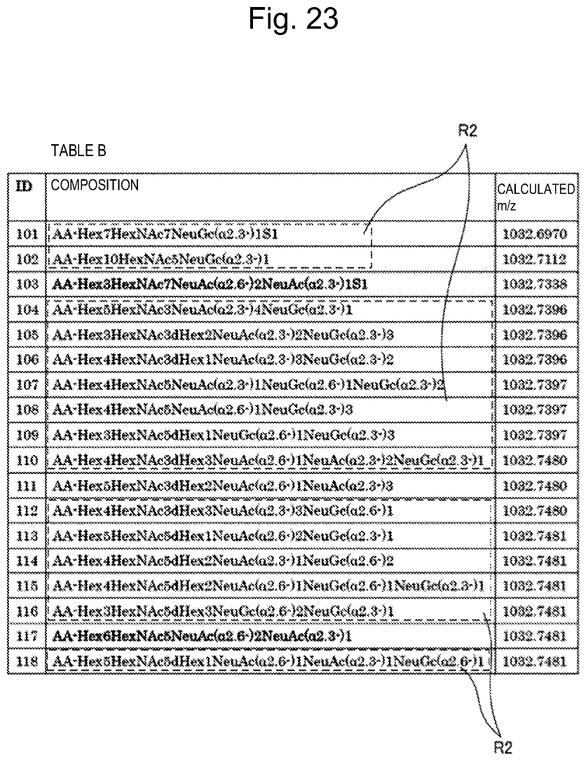

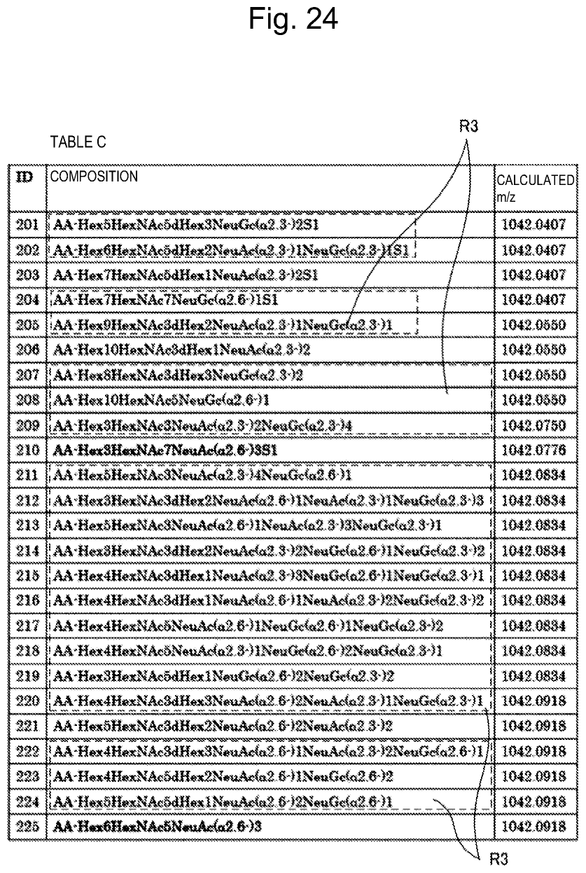

FIG. 22 is a table (Table A) showing glycan composition candidates obtained by the software based on m/z of glycan A. “ID” is a number associated with each composition. “Composition” is a composition of the candidate obtained by the search. “Calculated m/z” is a value of m/z theoretically calculated from each composition (the same applies to FIGS. 23 and 24 below). From 15 types of candidates shown in FIG. 22, candidates can be narrowed down as follows, using the oxonium ion information obtained by LC/MS of this example. First, since oxonium ions corresponding to sialic acid were detected in this example, candidates not containing sialic acid (IDs 1,2) can be excluded. Furthermore, since oxonium ions of sialic acid were detected at m/z 287 and 304 and m/z 315 and 333, it has been found to be NeuAc rather than NeuGc, and 9 candidates including Neu5Gc (candidates included in rectangle R1) can be further excluded. Among the remaining 4 types of candidates (IDs 3, 6, 11 and 15), considering that the ratio of the number of α2,6-sialic acid and the number of α2,3-sialic acid is 1:2 from the intensity ratio of each oxonium ion, the candidates can be narrowed down to two (IDs 3, 11). In addition to the above, analysis of the entire MS2 mass spectrum shows that the correct composition is ID 11.

Similar search was performed also for glycans B and C using the software as with glycan A. The m/z of glycan B was defined as 1032.7504, and the m/z of glycan C was defined as m/z 1042.0926.

FIG. 23 is a table (Table B) showing glycan composition candidates obtained by the software based on the m/z of glycan B. As shown in FIG. 23, 18 (IDs 101 to 118) hits were made as candidates for the composition of glycan B, but when candidates including NeuGc (candidates included in rectangle R2) were excluded, the number of candidates could be narrowed down to 3 (IDs 103, 111, 117). Furthermore, considering that the ratio of the number of α2,6-sialic acid and the number of α2,3-sialic acid was 2:1 from the intensity ratio of each oxonium ion, the candidates could be narrowed down to 2 types (IDs 103, 117).

FIG. 24 is a table (Table C) showing glycan composition candidates obtained by the software based on the m/z of glycan C. As shown in FIG. 24, 25 (IDs 201 to 225) hits were made as candidates for the composition of glycan C, but when candidates including Neu5Gc (candidates included in rectangle R3) were excluded, the number of candidates could be narrowed down to 5 types (IDs 203, 206, 210, 221 and 225). Furthermore, considering that only α2,6-sialic acid was contained from the intensity ratio of each oxonium ion, the candidates could be narrowed down to 2 types (IDs 210, 225).

In this way, by successfully utilizing oxonium ions generated from modified sialic acid, only peaks of acidic glycans or acidic glycopeptides having a specific linkage type can be visually displayed from a chromatogram of LC/MS or mass spectrum in which many peaks are detected. At the same time, in structural analysis of glycans, it is possible to narrow down candidates for glycan structure by utilizing the ratio of intensities of oxonium ions of modified sialic acid.

The disclosure of the following priority application is incorporated herein by reference.

Japanese Patent Application No. 2019-135344 (filed on Jul. 23, 2019)

### REFERENCE SIGNS LIST

1 . . . Mass Spectrometry Apparatus
10 . . . Liquid Chromatograph
20 . . . Mass Spectrometer
40 . . . Information Processing Unit
50 . . . Control Section
51 . . . Device Control Part
52 . . . Analysis Part
53 . . . Output Control Part
100 . . . Measurement Unit
521 . . . Data Acquisition Portion
522 . . . Chromatogram Creation Portion
523 . . . Mass Spectrum Creation Portion
524 . . . Calculation Portion
Di . . . Peaks of Dehydrated Oxonium Ions Derived from Isopropylamidated Sialic Acid
Dm . . . Peaks of Dehydrated Oxonium Ions Derived from Methylamidated Sialic Acid
Oi . . . Peaks of Non-Dehydrated Oxonium Ions Derived from Isopropylamidated Sialic Acid
Om . . . Peaks of Non-Dehydrated Oxonium Ions Derived from Methylamidated Sialic Acid

1. A mass spectrometry method comprising:
detecting, in a first mass spectrometry of a sample containing a glycan having a plurality of sialic acids each modified differently, a plurality of oxonium ions derived from each of the plurality of sialic acids; and
calculating relative values of intensities of the plurality of oxonium ions based on data obtained by the detection.

2. The mass spectrometry method according to claim 1, wherein
the plurality of sialic acids are amide-modified.

3. The mass spectrometry method according to claim 1, wherein
the first mass spectrometry is performed by tandem mass spectrometry in two or more stages.

4. The mass spectrometry method according to claim 1, comprising:
preparing a sample containing a glycan having sialic acid; and
modifying a plurality of sialic acids with each different linkage type contained in the glycan in a linkage type-specific manner,
wherein the first mass spectrometry of the sample containing the glycan having the plurality of modified sialic acids is performed.

5. The mass spectrometry method according to claim 4, wherein
α2,3-sialic acid, α2,8-sialic acid or α2,9-sialic acid, and α2,6-sialic acid are each modified differently.

6. The mass spectrometry method according to claim 4, comprising:
calculating a ratio of a number of a plurality of sialic acids having different linkage types in the glycan contained in the sample based on the relative values.

7. The mass spectrometry method according to claim 1, comprising:
performing chromatography of the sample before the first mass spectrometry.

8. The mass spectrometry method according to claim 7, comprising:
outputting an extracted ion chromatogram including a peak corresponding to at least one of the plurality of oxonium ions.

9. The mass spectrometry method according to claim 7, comprising:
performing mass separation of ions generated by ionization of the sample based on scanned m/z, performing dissociation of the mass-separated ions, and performing second mass spectrometry for detecting oxonium ions from the ions generated by the dissociation; and
obtaining at least one of a time during which a molecule containing a glycan from which the detected oxonium ion is derived is eluted in the chromatography and a mass of the molecule, based on a result of the second mass spectrometry.

10. A mass spectrometry apparatus comprising:
a data acquisition portion configured to acquire data obtained by detecting, in a first mass spectrometry of a sample containing a glycan having a plurality of sialic acids each modified differently, a plurality of oxonium ions each derived from the plurality of sialic acids; and
a calculation portion configured to calculate relative values of intensities of the plurality of oxonium ions based on the data.

11. A non-transitory computer readable medium recording a program for making a processor perform
a data acquisition process of acquiring data obtained by detecting, in a first mass spectrometry of a sample containing a glycan having a plurality of sialic acids each modified differently, a plurality of oxonium ions each derived from the plurality of sialic acids; and
a calculation process of calculating relative values of intensities of the plurality of oxonium ions based on the data.


SEQUENCE LISTING (2 sequences)

SEQ ID NO: 1 - protein, 6 aa, Artificial Sequence
  Digested peptide from alpha1 acid glycoprotein
NEEYNK

SEQ ID NO: 2 - protein, 16 aa, Artificial Sequence
  Digested peptide from haptoglobin
VVLHPNYSQVDIGLIK